(19) United States
(12) Patent Application Publication
(10) Pub. No.: US 2009/0156421 A1
(43) Pub. Date: Jun. 18, 2009

(54) ASSAYS AND METHODS FOR EVALUATING MULTIMERIC COMPLEXES

(75) Inventors: Xiaoke Yang, Boxborough, MA (US); Lori J. Fitz, Somerville, MA (US); Julie M. Lee, Somerville, MA (US); Jonathan Brooks, Wrentham, MA (US); Stanley F. Wolf, Arlington, MA (US)

Correspondence Address: LOWRIE, LANDO & ANASTASI, LLP;W2023, ONE MAIN STREET, SUITE 1100, CAMBRIDGE, MA, 02142, US

(73) Assignee: WYETH, Madison, NJ (US)

(21) Appl. No.: 12/266,126

(22) Filed: Nov. 6, 2008

Related U.S. Application Data
(60) Provisional application No. 61/002,142, filed on Nov. 6, 2007.

(51) Int. Cl. C40B 30/04; G01N 33/566

(52) U.S. Cl. 506/9; 436/501

(57) ABSTRACT

Assays, e.g., homogenous assays, and methods for identifying, quantifying and/or monitoring the formation and/or stability of a multimeric complex, e.g., a ternary complex are disclosed. The methods and assays of the invention can be used to identify and/or evaluate agents (e.g., proteins, peptides, antibody molecules, and small and large molecules) that interfere with and/or inhibit the formation of a multimeric complex (e.g., a ternary complex) or that disrupt a previously formed complex.

### CROSS REFERENCE TO RELATED APPLICATIONS

This application claims priority to U.S. Ser. No. 61/002,142, filed on Nov. 6, 2007, the contents of which are hereby incorporated by reference in their entirety.

### BACKGROUND

Screening assays, particularly high throughput screening (HTS) assays enable the testing of large numbers of compounds for activity in diverse areas of biology. Many screening methods currently available are limited by factors such as cost, speed, sensitivity, and reproducibility. In addition, currently available methods to screen for inhibitors of a target are limited to primarily monomeric target molecules or binary complexes. The availability of methods and assays for identifying modulators of multimeric complexes, such as ternary complexes, is more limited. Thus, the need exists for developing new and improved methods to identify and evaluate drug candidates that modulate an interaction of three or more members of a multimeric complex.

### SUMMARY

The present invention is based, at least in part, on the development of assays, e.g., homogenous assays, and methods for identifying, quantifying and/or monitoring the formation and/or stability of a multimeric complex, e.g., a ternary complex. In one embodiment, Applicants have developed homogenous assays that monitor the association of a ternary complex of a cytokine, e.g., interleukin-13 (IL-13) or a naturally-occurring IL-13 variant (e.g., IL-13R110Q), and its receptors (e.g., IL-13Rα1 and IL-4Rα, also referred to herein as “IL-13R1” or “IL-13 receptor,” or “IL-4R” or “IL-4 receptor”) using proximity-based detection methods, such as Time Resolved Fluorescence Resonance Energy Transfer (TR-FRET) and Surface Plasmon Resonance (SPR). The methods and assays of the invention can be used to identify and/or evaluate agents (e.g., proteins, peptides, antibody molecules, and small and large molecules) that interfere with and/or inhibit the formation of a multimeric complex (e.g., a ternary complex), or that disrupt a previously formed complex. In some embodiments, the formation of such complex results in a biological function, e.g., transduction of signal and/or a cellular response.

Accordingly, the invention provides a method, or an assay, for evaluating (e.g., detecting, quantifying and/or monitoring) the formation and/or stability of a multimeric complex, e.g., a ternary complex. The method includes providing a sample that includes at least three binding members under conditions that allow the formation of a multimeric complex to occur; detecting, quantifying and/or monitoring a change in the level of the multimeric complex (e.g., by detecting the formation and/or stability of the multimeric complex over a specified time interval, or in the presence of a test agent relative to a reference sample), thereby evaluating the formation and/or stability of the multimeric complex.

In a related aspect, a method, or assay, for identifying or evaluating an agent that modulates, e.g., decreases or increases, the formation and/or stability of a multimeric complex, e.g., a ternary complex, is disclosed. The method, or the assay, includes: contacting a sample that includes a first, second and third binding members with a test agent under conditions that allow the formation of the complex to occur; evaluating (e.g., detecting, quantifying and/or monitoring) the presence or amount of the complex in the sample contacted with the test agent relative to a reference sample (e.g., a control sample not exposed to the test agent; a control sample exposed to known modulator, e.g., inhibitor, of the complex; or a control sample exposed to an excess amount of an unlabeled binding member of the complex). A change (e.g., an increase or a decrease) in the level of the complex in the presence of the test agent, relative to the level of the complex in the reference sample, indicates that said test agent affects (e.g., increases or decreases) the formation and/or stability of said complex. In some embodiments, the test agent decreases complex formation by, e.g., about 1.5, 2, 5, 10 fold or higher, relative to a reference sample.

In another aspect, the invention provides a method of evaluating or selecting a multimeric complex binding agent, e.g., an anti-IL13 ternary complex binding agent. The method includes:

providing a first sample that includes the multimeric complex binding agent (e.g., a sample or batch sample containing an anti-IL13 ternary complex binding agent);

contacting the first sample with a second sample that includes a multimeric complex, or one or more members of the multimeric complex;

evaluating (e.g., detecting, quantifying and/or monitoring) at least one parameter of the assembly, stability and/or function of the multimeric complex in the presence of the multimeric complex binding agent;

(optionally) comparing the at least one parameter with a reference value, to thereby evaluate or select the multimeric complex binding agent.

The comparison can include determining if the at least one parameter has a pre-selected relationship with the reference value, e.g., determining if it falls within a range of the reference value (either inclusive or exclusive of the endpoints of the range); is equal to or greater than the reference value. In certain embodiments, if the at least one parameter meets a pre-selected relationship, e.g., falls within the reference value, the multimeric complex binding agent is selected. In other embodiments, the assays, methods, or an indication of whether the pre-selected relationship between the at least one parameter and a reference value is met, is recorded or memorialized, e.g., in a computer readable medium. Such methods, assays or indications of meeting pre-selected relationship can be listed on the product insert, a compendium (e.g., the U.S. Pharmacopeia), or any other materials, e.g., labeling that may be distributed, e.g., for commercial use, or for submission to a U.S. or foreign regulatory agency.

In some embodiments, the multimeric complex binding agent is an antibody molecule that binds to a cytokine ternary complex, or a member thereof (e.g., a cytokine receptor or a co-receptor). For example, the test agent can be an antibody molecule that binds to the IL-13 ternary complex, or a member thereof (e.g., IL-13, an IL-13 receptor and/or an IL-4 receptor). The antibody molecule can be obtained, e.g., from a sample batch of an antibody culture. Methods disclosed herein can be useful from a process standpoint, e.g., to monitor or ensure batch-to-batch consistency or quality.

In embodiments, a decision or step is taken depending on whether the at least one parameter meets the pre-selected relationship (e.g., falls within the range provided for the reference value). For example, the IL-13 complex binding agent, e.g., the anti-IL13 complex antibody molecule, can be classified, selected, accepted, released (e.g., released into commerce) or withheld, processed into a drug product, shipped, moved to a new location, formulated, labeled, packaged, sold, or offered for sale.

The methods and assays disclosed herein can be used to identify or test modulators of a signaling or biological activity, e.g., a cytokine signaling or biological activity. For example, test agents that modulate, e.g., inhibit, IL-13 signaling can be identified using the methods disclosed herein by identifying agents that (a) modulate, e.g., interfere with, the formation and/or stability of a binary complex of IL-13 (e.g., by modulating, e.g., interfering with, an interaction between the cytokine and its receptor (e.g., IL-13 and IL-13Rα1)) and/or (b) by modulating, e.g., interfering with, the formation and/or stability of an IL-13 ternary complex (e.g., by interfering with the interaction between one or two members of the binary complex and a co-receptor (e.g., IL-4Rα).

Additional embodiments of the aforesaid methods and assays may include one or more of the following features:

In certain embodiments, the multimeric complex includes three, four, five or more binding members. For example, a binding member of the multimeric complex can include a peptide, a polypeptide (e.g., a cytokine, a chemokine, or a growth factor in association with at least one, typically, two corresponding receptors), a large or small molecule (e.g., a macrolide or a polyketide in association with at least one, typically two macrolide- or polyketide-associated proteins), or any combination thereof. In one embodiment, the multimeric complex includes a first binding member, e.g., a ligand or an activator of the second and/or third binding member (e.g., a cytokine); a second binding member, e.g., a ligand receptor (e.g., a cytokine receptor), and a third binding member, e.g., a ligand co-receptor (e.g., a cytokine receptor subunit that interacts with the cytokine receptor and/or the cytokine). Examples of multimeric complexes that can be evaluated using the methods and assays of the invention include but are not limited to, for example, complexes of an interleukin and its receptors chosen from one of more of: interleukin 2 (IL-2), interleukin 6 (IL-6), interleukin 4 (IL-4), interleukin 5 (IL-5), interleukin 10 (IL-10), interleukin-13, interleukin 15 (IL-15), interleukin 21 (IL-21) and/or interleukin 22 (IL-22). For example, the multimeric complex can be a ternary complex that includes IL-13 as a first binding member, an IL-13 receptor α1 (IL-13Rα1) as a second binding member, and an IL-4 receptor (IL-4Rα) as a third binding member.

In certain embodiments, the methods or assays of the invention can be used to evaluate at least one parameter of the assembly, stability and/or function of the multimeric complex, including but not limited to, kinetics of complex association or dissociation, binding affinity, steady-state binding parameters, and/or effective or inhibitory concentrations (e.g., k_{d}, kₒₙ, k_{off}, EC₅₀ and/or IC₅₀).

In other embodiments, the method, or assay, further includes contacting the multimeric complex with a known inhibitor of the complex, or an excess amount of one or more of the binding members (e.g., an excess amount of unlabeled binding member) to detect the inhibition of complex formation and/or dissociation rate of the complex. Such step can be carried out in the absence or presence of a test agent to detect the effect of the test agent on the inhibition and/or dissociation rate of the complex. A change in binding (e.g., complex formation) and/or activity, in the presence or absence of the test agent, is indicative that the test agent modulates the formation and/or dissociation of the complex, and/or modulates an interaction of the known inhibitor with the complex.

In other embodiments, the method, or assay, further includes the step(s) of comparing binding of the test agent to the complex to the binding of the known compound to the complex. The method, or assay, can additionally, optionally, include detecting the interaction (e.g., binding) of the test agent to one or more of the binding members, in complexed or uncomplexed form.

In other embodiments, the method, or assay, further includes the step(s) of recording or memorializing, e.g., in a computer readable medium, one of more of the methods, assays or parameters disclosed herein. Such information can be listed on a product insert, a compendium (e.g., the U.S. Pharmacopeia), or any other materials, e.g., labeling that may be distributed, e.g., for commercial use, or for submission to a U.S. or foreign regulatory agency.

Test agents can be, for example, a polypeptide (e.g., an antibody molecule, a soluble receptor, or a binding domain fusion protein), large or small molecule (e.g., a naturally-occurring molecule or a synthetic molecule (e.g., a member of a combinatorial library). In one embodiment, the test agent interacts, e.g., binds to, the multimeric complex, or one or more of the binding members of the multimeric complex. Test agents can be produced recombinantly; chemically (e.g., small molecules, including peptidomimetics); or as a natural product of bacteria, actinomycetes, yeast or other organisms. In one embodiment, the test agent binds to an IL-13 ternary complex, or a member thereof (e.g., an IL-13 receptor or an IL-4 receptor). For example, the test agent can be an antibody molecule that binds to the IL-13 ternary complex, or a member thereof (e.g., IL-13, an IL-13 receptor and/or an IL-4 receptor). In embodiments, the test agent is a collection or library of multimeric complex binding agents, e.g., a collection of antibody molecules, variant molecules, small or large molecules, or receptor fusions. In other embodiments, the test agent is a sample obtained from a sample batch of a production or manufacturing pool (e.g., an antibody culture). Accordingly, test agents evaluated by the methods and assays disclosed herein can be used to monitor or ensure batch-to-batch consistency or quality.

A variety of assay formats will suffice and, in light of the present disclosure, those not expressly described herein will nevertheless be understood by one of ordinary skill in the art. Assay formats which approximate such conditions as formation of protein complexes may be generated in many different forms, and include assays based on cell-free systems, e.g., purified proteins or cell lysates, as well as cell-based assays which utilize intact cells and in vivo assays. Binding assays can be used to detect compounds that inhibit or potentiate one or more interactions between binding members of the complex.

In certain embodiments, the present invention provides a reconstituted preparation including one or more binding members. In one embodiment, the binding members of the complex are added simultaneously in a sample, e.g., a reaction mixture. In other embodiments, the sample is prepared by adding the binding members sequentially in any order, e.g., forming a mixture of the first member (e.g., a cytokine) with a second member (e.g., a cytokine receptor), and adding the third member (e.g., a cytokine co-receptor). In another embodiment, a mixture of the second member (e.g., a cytokine receptor) and the third member (e.g., a cytokine co-receptor) is formed, followed by addition of the first member (e.g., a cytokine). In yet other embodiments, a mixture of the first member (e.g., a cytokine) and the third member (e.g., a cytokine co-receptor) is formed, followed by addition of the second member (e.g., a cytokine receptor). Any order or combination of the binding members can be used. Assays of the present invention include labeled in vitro protein-protein binding assays, immunoassays for protein binding, and the like, as described in more detail below. In one embodiment, the sample is a cell lysate or a reconstituted system (e.g., cell a membrane or a soluble component (e.g., a soluble fragment of a receptor or a receptor fused to a heterologous moiety, e.g., a receptor fused to an immunoglobulin fragment)). The reconstituted complex can include a reconstituted mixture of at least semi-purified proteins. In certain embodiments, assaying in the presence and absence of a test agent, can be accomplished in any vessel suitable for containing the reactants. Examples include microtitre plates, test tubes, and micro-centrifuge tubes. Alternatively, the sample can include cells in culture, e.g., purified cultured or recombinant cells, or in vivo in an animal subject.

In certain embodiments, the methods and assays of the invention detect a change in multimeric complex formation and/or stability by detecting one or more of: a change in the binding or physical formation of the complex itself, e.g., by biochemical detection, affinity based detection (e.g., Western blot, affinity columns), immunoprecipitation, fluorescence resonance energy transfer (FRET)-based assays (e.g., FRET or Time Resolved FRET assays (TR-FRET)), surface plasmon resonance (SPR), spectrophotometric means (e.g., circular dichroism, absorbance, and other measurements of solution properties); a change, e.g., an increase or a decrease, in signal transduction, e.g., phosphorylation and/or transcriptional activity; a change, e.g., increase or decrease, cell function. In embodiments where the ternary complex includes IL-13 and IL-13 receptors, one or more of the following IL-13-associated activities can be evaluated: induction of CD23 expression; production of IgE by B cells; phosphorylation of a transcription factor, e.g., STAT protein (e.g., STAT6 protein); antigen-induced eosinophilia in vivo; antigen-induced bronchoconstriction in vivo; drug-induced airway hyperreactivity in vivo; eotoxin levels in vivo; and/or histamine release by basophils. In one embodiment, the test agent is identified and re-tested in the same or a different assay. For example, a test agent is identified in an in vitro or cell-free system, and re-tested in an animal model or a cell-based assay. Any order or combination of assays can be used. For example, a high throughput assay can be used in combination with an animal model or tissue culture.

In embodiments where the methods and assays detect a change in multimeric complex formation and/or stability by FRET and/or TR-FRET, two or more of the binding members of the multimeric complex are labeled with fluorescent molecules having the proper emission and excitation spectra, such that when brought into close proximity with one another emit a detectable fluorescent signal. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. FRET-based assays are described in more detail herein.

Assays or detection methods can be used to identify test agents that modulate, e.g., interfere with, the formation and/or stability of a binary and/or the ternary IL-13 complex. For example, this method may be used to identify test agents that modulate, e.g., interfere with, an interaction between (a) IL-13 and IL-13Rα1, (b) IL-4Rα and IL-13Rα1, (c) IL-13 and IL-4R, as well as (c) test agents that modulate, e.g., interfere, with an interaction among IL-13, IL-13Rα1 and IL-4Rα, by modulating an interaction between two or more of these binding agents. For example, an assay that detects an interaction between IL-4R and either IL-13 or IL-13Rα1 can be used to screen for inhibitors that reduce the formation and/or stability of the ternary IL-13 complex. Without being bound by theory, IL-13 is believed to interact initially with IL-13Rα1 forming a binary complex, which binary complex then interacts with IL-4Rα. The trimeric complex of IL-13, IL-13Rα1 and IL-4R was found to have increased affinity for IL-13 (Kd from 6.0 nM to 0.28 nM). Test agents that modulate, e.g., interfere with, one or more of these interactions can be evaluated using the methods and assays described herein. The assays and methods described herein may be adapted to detect formation and/or stability of other multimeric complexes, e.g., other ternary complexes, including but not limited to, for example, complexes of an interleukin and its receptors chosen from one of more of: IL-2, IL-4, IL-5, IL-6, IL-10, IL-15, IL-21 and/or IL-22.

In one exemplary embodiment where an IL-13 multimeric complex is evaluated, at least two of the binding members can be labeled for FRET detection. One of skill will appreciate that the methods and assays described herein can be practiced by labeling the at least two binding members with any combination of suitable FRET acceptor and donor. In one embodiment, the first and the second or third binding members (e.g., a IL-13 and IL-13R or IL-4Rα) are labeled for FRET detection, for example, by labeling IL-13 with a suitable FRET donor and IL-13R or IL-4Rα with a suitable FRET acceptor. For example, IL-13 may be labeled (e.g., directly labeled) with europium chelate (Eu) and IL-13R or IL-4Rα may be labeled (e.g., directly labeled) with Alexa Fluor 647 (FL647) or Cy5, using the methods described herein. In another embodiment, the second and third binding members (e.g., a IL-13Rα1 and IL-4Rα, respectively) may be labeled with a suitable FRET donor and acceptor. For example, IL-13Rα1 may be labeled (e.g., directly labeled) with europium chelate (Eu) and IL-4R may be labeled (e.g., directly labeled) with Alexa Fluor 647 (FL647) or Cy5, using the methods described herein. Such methods and assays may be used to identify test agents that interfere with the formation of a ternary complex. For example, these methods and assays may be used to identify test agents that interfere with the interaction between the binary complex of IL-13 and IL-13Rα1, and/or an interaction between the IL-13/IL-13Rα1 binary complex and IL-4R. One of skill in the art will appreciate that this method may also be practiced to achieve the same result by labeling IL-13Rα1 with a suitable FRET acceptor and IL-4R with a suitable FRET donor, or other combinations thereof.

In some embodiments, methods and/or assays as described herein can be practiced using combinations of the above described (a) IL-13 and IL-4R and (b) IL-13Rα1 and IL-4R labeling methods. For example, labeling of the binders members in (a), practiced alone, will identify modulators, e.g., inhibitors, of IL-13 binary and ternary complex formation. Labeling of the binders members in (a), practiced alone, will not allow a modulator, e.g., inhibitor, of an IL-13 binary complex to be distinguished from a modulator, e.g., inhibitor, of an IL-13 ternary complex. Labeling of the binders members in (b), practiced alone, will identify inhibitors of the IL-13 binary and ternary complex formation. For example, labeling of the binding members in (b), practiced alone, will allow identification of a test agent that modulates the association between IL-4R and IL-13Rα1. However, labeling of the binding members in (b), practiced alone, will also identify a test agent that modulates the association between IL-13 and IL-13Rα1, as IL-4R is believed to not bind to IL-13Rα1 in the absence of IL-13. Combination of labeling of the binding members in (a) and (b), however, will allow the identification of one or more of: a test agent that modulates formation and/or stability of a binary and ternary complex; a test agent that modulates formation and/or stability of a binary complex; and/or a test agent that modulates formation and/or stability of a ternary complex. For example, if a test agent interferes with binary complex formation and/or stability both (a) and (b), FRET signaling will be decreased. If a test agent interferes with both binary and ternary complex formation either the FRET signaling for (a) will be reduced, and/or the FRET signaling for (a) and (b) will be reduced.

In some embodiments, the screening assays described herein, e.g., a TR-FRET assay, may be performed in vitro using isolated binding members. In such a system, each component of the screen may be added separately in wells of a multi-well plate, for example 96, 384, and 1536-well plates. In some embodiments, the multimeric complex will be allowed to form prior to the addition of the test agent to be screened. In other embodiments, the members of the complex and the test agent will be added together, e.g., at the same time or simultaneously, with one or more of the members of the complex. In some embodiments, the screening assay evaluates a plurality of different test agents, at a fixed or a range of concentrations. In some embodiments, the screening assay will screen a known or previously identified inhibitor of the complex.

In some embodiments, the methods and assays described herein may be performed using TR-FRET. In such a system a detected decrease in the TR-FRET signal, e.g., a 0.5%, 1%, 1.5%, 3%, 5%, 10%, 20%, or higher is indicative that a test agent is an inhibitor of the complex. In some embodiments, the percent decrease will be compared to a reference value, e.g., a previously established percent decrease for the same molecule, for example, when validating a molecule. In some embodiments, a reference value, e.g., a threshold percent decrease, will be established prior to the screen. Test agents that meet said reference or threshold value are considered to be effective.

In other embodiments, the methods and assays described herein may be performed in vivo, using for example Bioluminescence Resonance Energy Transfer (BRET). In such a system, the members of the multimeric complex may be overexpressed as fusion proteins within a cell. The fusion may be a detectable label, e.g., a fluorophore selected from Table 2, and at least two of the members of the complex will be labeled. In some embodiments, the complex is labeled indirectly using, for example labeled antibodies. In such a system, the components of the ternary complex may be overexpressed proteins, e.g., fusion proteins containing a detectable marker, e.g., a six histidine tag or an Xpress™ epitope, that can be detected (i.e., probed) with a commercially available antibody. In some embodiments, the components of the ternary complex may be endogenous proteins that are probed with at least two protein specific antibodies with labels that are capable of BRET. In such a system, a detected decrease in the BRET signal, e.g., a 0.5%, 1%, 1.5%, 3%, 5%, 10%, 20%, and above will be considered a positive indication that a screened molecule is an inhibitor of a ternary complex.

In other embodiments, the method, or assay, can be performed in a cell. The method includes providing a step based on proximity-dependent signal generation, e.g., a two- or three-hybrid assay that includes a first binding member (e.g., a cytokine), a fusion protein (e.g., a fusion protein comprising a portion of the second binding member (e.g., a cytokine receptor)), and another fusion protein (e.g., a fusion protein comprising a portion of the third binding member (e.g., a cytokine co-receptor), using cells in culture, e.g., purified cultured or recombinant cells. The method, or assay includes: contacting the two- or three-hybrid assay with a test agent, under conditions wherein said hybrid assay detects a change in the formation and/or stability of the complex, e.g., the formation of the complex initiates transcription activation of a reporter gene.

In other embodiments, methods and assays for detecting complex formation include the step of immobilizing one or more of the binding members of the complex to a solid support, e.g., a matrix or a bead. Immobilization of the one or more binding members can facilitate separation of the complex from uncomplexed forms of one of the members of the complex, as well as to accommodate automation of the assay. Affinity matrices or beads are described herein that contain the ligand (or other members of the complex) that permits other components of the complex to be bound to an insoluble matrix. In embodiments, a test agent is incubated under conditions conducive to complex formation; washing off the support, e.g., beads, to remove any unbound interacting binding member; and determining the amount of bound binding members in the complex, by, e.g., quantifying the amount of matrix bead-bound binding member directly (e.g., beads placed in scintillant if one or more of the bound members are radiolabeled), or in the supernatant after the complexes are dissociated, e.g., when microtitre plate is used. Alternatively, after washing away unbound protein, the complexes can be dissociated from the matrix, separated by SDS-PAGE gel, and the level of interacting binding member found in the matrix-bound fraction quantitated from the gel using standard electrophoretic techniques.

In yet another aspect, the invention features a multimeric binding agent, e.g., an anti-IL13 complex binding agent, identified or evaluated by the methods or assays described herein. In embodiments, the binding agent is other than 13.2, MJ2-7 and C65 (or humanized versions thereof). Compositions, e.g., pharmaceutical compositions, that include the multimeric binding agents of the invention and a pharmaceutically-acceptable carrier are disclosed. In one embodiment, the compositions include the compounds of the invention in combination with one or more agents, e.g., therapeutic agents. In one embodiment, the second agent is an immunomodulator, e.g., an immunosuppressant. Examples of immunomodulators that can be used in combination with the agents identified herein include one or more of: TNF antagonists (e.g., a soluble fragment of a TNF receptor, e.g., p55 or p75 human TNF receptor or derivatives thereof, e.g., 75 kd TNFR-IgG (75 kD TNF receptor-IgG fusion protein, ENBREL™)); TNF enzyme antagonists, e.g., TNFα converting enzyme (TACE) inhibitors; muscarinic receptor antagonists; TGF- [symbol] antagonists; interferon gamma; perfenidone; chemotherapeutic agents, e.g., methotrexate, leflunomide, or a sirolimus (rapamycin) or an analog thereof, e.g., CCI-779; COX2 and cPLA2 inhibitors; NSAIDs; immunomodulators; p38 inhibitors, TPL-2, Mk-2 and NFKB inhibitors. In certain embodiments, the amount of the agent administered present in the combination composition is lower than the amount of the agent present in compositions administered individually.

In another aspect, the invention features method of treating a disorder or condition associated with aberrant activity or expression of one or more members of a multimeric complex in a subject having, or being at risk of having, the disorder or condition. The method includes administering a multimeric binding agent to the subject, wherein the multimeric binding agent has at least one parameter of complex formation and/or stability evaluated by the methods or assays disclosed herein. The at least one parameter can be evaluated prior to or after the administration step.

In another aspect, the invention features method of treating a disorder or condition associated with aberrant activity or expression of one or more members of a multimeric complex in a subject having, or being at risk of having, the disorder or condition. The method includes:

instructing a caregiver or a patient that a multimeric complex binding agent, e.g., an anti-IL13 complex antibody, has at least one parameter of complex formation and/or stability evaluated by the methods or assays disclosed herein,

administering the binding agent to the patient. The administration step can be performed by the patient directly, e.g., self-administration, or by another party, e.g., a caregiver.

In yet another aspect, the invention provides methods and assays to identify previously unidentified components within a multimeric complex. The methods, or assays, include: (1) detectably identifying a library of candidate binding member (e.g., labeling a library of candidate members with a FRET donor); (2) detectably identifying at least one known member of the complex (e.g., labeling at least one known member of the complex with a FRET acceptor); (3) contacting said identified library with said identified at least one member of the complex, under conditions that allow an interaction to occur, wherein the interaction of the library member with the at least one member of the complex results in a detectable signal; (4) detecting the signal generated, e.g., by performing FRET or TR-FRET analysis. A change, e.g., an increase, in the signal generated upon association of the library member with the at least one member of the complex is indicative the association and/or complex formation. The method, or assays, can optionally include the step of identifying and/or obtaining the complex.

In another aspect, the invention provides reagents for carrying out the aforesaid assays and methods, including but not limited to, antibody molecules that recognize one or more binding members of the complexes described herein; as well as host cells and/or vectors comprising one or more nucleic acids encoding one or more of the polypeptide members of the complex disclosed herein.

In another aspect, the invention features a kit that includes a multimeric complex binding agent or an assay disclosed herein, and instructions for use. In certain embodiments, the multimeric complex binding agent included in the kit is or has at least one parameter of complex formation and/or stability evaluated by the methods or assays disclosed herein.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Typically, exemplary degrees of error are within 20 percent (%), preferably within 10%, and more preferably within 5% of a given value or range of values. In the context of residues in nucleic acid or amino acid sequences, “about” refers to variation of up to 5 residues (e.g., 5, 4, 3, 2, or 1 residue variation from a disclosed sequence or a particular residue in a disclosed sequence).

The details of one or more embodiments of the invention are set forth in the accompanying drawings and the description below. Other features, objects, and advantages of the invention will be apparent from the description and figures, and from the claims.

### DESCRIPTION OF DRAWINGS

FIGS. 1A-1D are line graphs showing surface plasmon resonance (SPR) measurements of IL-13 and IL-13R110Q binding to IL-13Rα1 in the presence and absence of IL-4R. FIG. 1A. and FIG. 1B show response units monitored in real time for various concentrations (0-40 nM) of IL-13 (A) and IL-13R110Q (B) injected over a heterogeneous IL-13Rα1 coated sensor chip surface. FIG. 1C and FIG. 1D show response units monitored in real time for various concentrations (0-40 nM) of IL-13 (A) and IL-13R110Q (B) injected over a heterogeneous IL-13Rα1 and IL-4R coated sensor chip surface. For each FIG the data, shown in the black wavy lines, were fit to a heterologous ligand model in BiaEval v4.1, overlayed with a solid red line. Data shown for each concentration are triplicate measurements. Each data set is representative of at least 3 independent experiments.

FIGS. 2A-2B are line graphs showing SPR measurements of IL-4R binding kinetics to the IL-13/IL-13RI1 binary complex. Response units monitored in real time for various dilutions of IL-4R (0 to 400 nM) after injection on either (A) IL-13/IL-13Rα1 or (b) IL-13R110Q/IL-13Rα1 binary complex coated on the surface of a heterogeneous sensor chip surface. For each graph the data, shown in the black wavy lines, are triplicate measurements for each concentration. The calculated fit from a 1:1 model using BiaEval software v4.1 is shown using a solid red line. Each data set is representative of 3 independent experiments.

FIG. 3 is a schematic representation of TR-FRET binary assay (assay 1). A binary TR-FRET complex was formed using Eu-IL-13 and Cy5-IL-13Rα1. Measurement conditions were; excitation at 345 nM, detection at 615 nM to monitor the europium signal, and detection at 665 nM to monitor TR-FRET.

FIGS. 4A-4B is a schematic representation of TR-FRET ternary assays (assays 1 and 2). A ternary TR-FRET complex was formed using Eu-IL-13 and IL-4R-FL647. Unlabeled IL-13RI1 is added for the ternary complex formation (FIG. 4A). A second ternary TR-FRET assay format is shown in FIG. 4B using Eu-IL-13 and IL-13R-Cys5. Unlabeled IL-4R is added for the ternary complex formation. Measurement conditions were excitation at 345 nM, detection at 615 nM to monitor the europium signal, and detection at 665 nM to monitor TR-FRET.

FIGS. 5A and 5B are line graphs showing dissociation constants of Cy5-IL-13Rα1 in the absence of IL-4R (A) or in the presence of IL-4R (B) measured using TR-FRET assay 1. Increasing concentrations of Cy5-IL-13Rα1 were added to 10 nM Eu-IL-13. IL-4R was added at 500 nM. Dissociation constants were calculated from IC50 values using Equation (1). All experiments were done in duplicate and the data points were an average of two.

FIGS. 6A-6F are a series of line graphs showing binding comparisons of IL-13, IL-13R110Q, and IL-13Rα1 in the formation of the binary and ternary complex measured using TR-FRET assay 1. FIGS. 6A and 6B are line graphs showing TR-FRET ratio formed by 10 nM each Eu-IL-13 and Cy-5-IL-13Rα1 with increasing concentrations of unlabeled (A) IL-13 or (B) IL-13R110Q to disrupt the binary complex. FIGS. 6C and 6D are line graphs showing TR-FRET ratio formed by 10 nM of Eu-labeled IL-13 and Cy-5-labeled IL-13Rα1 plus 500 nM of IL-4R and increasing concentrations of unlabeled (C) IL-13 (D) or IL-13R110Q to disrupt the ternary complex. FIGS. 6E and 6F are line graphs showing TR-FRET ratio formed by 10 nM each Eu-IL-13 and Cy5-IL-13Rα1 monitored after adding increasing concentrations of unlabeled IL-13Rα1 in the (E) absence and (F) presence of 500 nM of IL-4R. For all of FIGS. 6A-6F, dissociation constants were calculated from IC50 values using Equation (2). All experiments were done in duplicate and the data points were an average of two.

FIGS. 7A and 7B are line graphs showing data generated using TR-FRET assay 2. TR-FRET signal for (A) 20 nM each, Eu-IL-13 and unlabeled IL-13Rα1 and increasing concentrations of IL-4R-FL647 (0-1100 nM); or (B) 40 nM of Eu-IL-13 and 400 nM of IL-4R-FL647 and increasing concentrations of unlabeled IL-13Rα1 (0-200 nM). IL-4R binding affinity was calculated using the direct binding method described herein. All experiments were done in duplicate and the data points were averaged.

FIGS. 8A and 8B are line graphs showing TR-FRET assay 2 validation using IL-13 and two distinct IL-13 antibodies. FIG. 8A is a line graph depicting the kinetics of a TR-FRET complex formed with 20 nM Eu-IL-13, 500 nM IL-4R-FL647 and 20 nM IL-13Rα1, which was monitored in kinetic mode after (red) the addition of 3.0 μM unlabeled IL-13 compared to (black) a positive control with no addition of unlabeled IL-13 or compared to (blue) a no TR-FRET control with labeled IL-13 and no IL-13Rα1. FIG. 8B is a line graph depicting the kinetics of TR-FRET complex formed with 20 nM Eu-IL-13, 25 nM IL-13Rα1 and 200 nM of IL-4R-FL647, which was monitored in kinetic mode after the addition of 300 nM of the two indicated antibodies against IL-13, humanized antibody 13.2, shown in red or Ab026, shown in blue compared to a positive control with no addition of antibody, shown in black.

FIGS. 9A-9B are graphs depicting the kinetics of IL-13 binding in the absence (FIG. 9A) or the presence (FIG. 9B) of an anti-IL13 antibody. The k_{d} value changes from about 7 nM (FIG. 9A) to 5 nM (FIG. 9B). The presence of the antibody does change the intensity of the TR-FRET signal.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety.

### DETAILED DESCRIPTION

The present invention is based, at least in part, on the development of assays, e.g., homogenous assays, and methods for identifying, quantifying and/or monitoring the formation and/or stability of a multimeric complex, e.g., a ternary complex. In one embodiment, Applicants have developed homogenous assays that monitor the association of a ternary complex of a cytokine, e.g., IL-13 or a naturally-occurring IL-13 variant (e.g., IL-13R110Q), and its receptors (e.g., IL-13Rα1 and IL-4Rα) using proximity-based detection methods, such as Time Resolved Fluorescence Resonance Energy Transfer (TR-FRET) and Surface Plasmon Resonance (SPR). Assays targeting the interaction between IL-4R and the binary complex of IL-13 and IL-13Rα1 have been developed. The assays developed herein have been corroborated with two antibody inhibitors of the IL-13 complex that are known to block epitopes located on IL-13 that interact with either IL-13Rα1 or IL-4R. Accordingly, the methods and assays of the invention can be used to evaluate at least one parameter of the assembly, stability and/or function of the multimeric complex, including but not limited to, kinetics of complex association or dissociation, binding affinities and/or steady-state binding parameters (e.g., k_{d}, kₒₙ, k_{off}, and/or IC₅₀). Such methods and assays are useful for identifying agents that modulate, e.g., inhibit or increase, the formation and/or stability of a multimeric complex, e.g., a ternary complex.

### Assays to Identify Modulators of Multimeric Complexes

Screening assays can be generally categorized as heterogeneous and homogeneous assays. Heterogeneous assays differ from homogenous assays in that they generally require the use of a solid phase and one or more washing steps to carry out the assay. Typically, the components of a homogeneous assay are present during measurement, and the reactions occur generally in solution without a solid-phase. Because homogeneous assays do not require wash steps or a solid phase, they are typically faster, easier, and more economical to perform.

In general, in a heterogeneous assay, at least one molecule in a sample is labeled with a detectable signal, e.g., a marker group. The amount of the analyte molecule to be examined is evaluated by measuring the detectable signal. Determination of the detectable signal, e.g., the amount of the marker group, present in the sample is of use only when bound and unbound labeled binding partners have been separated, for example, by means of at least one round of a suitable washing step. The washing step is typically performed prior to determination of the marker. Exemplary marker groups include, photon effects (e.g., a luminescent or a fluorescent mechanism), colorimetric effects, radioactive effects, and scattered light effects. Heterogeneous assays include but are not limited to, for example, enzyme immunoassays, enzyme-linked immunoassays (ELISA), surface plasmon resonance (SPR), and DNA hybridization techniques where a solid phase is involved.

In contrast, in a homogeneous assay, test conditions are selected such that a detectable signal change occurs in solution. This signal change is dependent on the concentration of the analyte molecule (e.g., an altered substrate, a metabolite, and a complex of two or more molecules) present in the sample. For example, the signal change can be used to determine the amount of the analyte molecule present. Exemplary detectable signal changes include turbidity effects, photon effects (e.g., a luminescent or a fluorescent mechanism), calorimetric effects, radioactive effects, and scattered light effects. Homogeneous assays include but are not limited to, for example, cloned enzyme donor immunoassays (CEDIA, Microgenics Inc., USA), scintillation proximity assays (SPA, Amersham, UK), luciferase assays (Promega, USA), fluorescence techniques (e.g., fluorescence intensity, fluorescence polarization assays (FPIA, Syva Co., USA), fluorescent linked immunosorbent assay (FLISA, Applied Biosystems, USA)), time-resolved fluorescence (PerkinElmer, USA), fluorescence correlation spectroscopy, fluorescence resonance energy transfer (FRET), quenched autoligation-FRET (QFRET), and Bioluminescence Resonance Energy Transfer (BRET) based assays).

Fluorescent molecules are now the most commonly used markers for screening methods that require the use of detectable marker groups. Fluorescent techniques offer several advantages over previously used techniques such as radiolabeling, for example fluorescent techniques are easily adapted for homogeneous assays and can be excited thousands of times, without the hazards associated with radioactive techniques.

Accordingly, the present invention provides at least in part methods and assays to identify or characterize agents (e.g., proteins and peptides, antibody molecules, small or large molecules) that interfere with and/or inhibit the formation of a multimeric complex (e.g., a ternary complex) or that disrupt a previously formed complex. As used herein, the term “multimeric complex” refers to an association or binding (e.g., a covalent or non-covalent association or binding) of three or more binding members. In certain embodiments, the multimeric complex includes three, four, five or more binding members. In some embodiments, the formation of such complex results in a biological function, e.g., transduction of signal and/or a cellular response. The methods described herein, however, do not exclude the possibility that additional molecules or factors (i.e., in addition to the binding members of the complex) that may be part of the complex, e.g., as auxiliary factors. Such additional molecules or factors may be included in the assays or methods described herein.

As used herein, the terms “binding” and “complex formation” refer to a direct or indirect association between two or more molecules, e.g., polypeptides, among others. Direct associations may include, for example, covalent, electrostatic, hydrophobic, ionic and/or hydrogen-bond interactions under physiological conditions. Indirect associations include, for example, two or more molecules that are part of a complex, but do not have a direct interaction. In some embodiments, the association between the molecules is sufficient to maintain a stable complex under physiological conditions.

Examples of the multimeric complexes that can be evaluated using the methods and assays of the invention include but are not limited to, for example, complexes of an interleukin and its receptors chosen from one of more of: interleukin 2 (IL-2), interleukin 4 (IL-4), interleukin 5 (IL-5), interleukin 6 (IL-6), interleukin 10 (IL-10), interleukin-13, interleukin 15 (IL-15), interleukin 21 (IL-21), and/or interleukin 22 (IL-22). It shall be understood that the present invention can be practiced using variants of the aforesaid cytokines and their receptors. As used herein, a “variant” of a polypeptide, or fragment thereof, such as, for example, a variant of a cytokine includes chimeric proteins, labeled proteins (e.g., fluorescently labeled), fusion proteins, mutant proteins, proteins having similar (e.g., substantially similar) sequences (e.g., proteins having amino acid substitutions (e.g., conserved amino acid substitutions), deletions, insertions, amino acid sequences at least about 85%, 90%, 95% or more identical to a naturally-occurring sequence), protein fragments, mimetics, so long as the variant has at least a portion of an amino acid sequence of a native protein, or at least a portion of an amino acid sequence of substantial sequence identity to the native protein. A “functional variant” includes a variant that retains at least one function of the native protein, e.g., retains the ability to interact with and/or form a complex as described herein. As used herein, a “chimeric protein” or “fusion protein” is a fusion of a first amino acid sequence encoding a polypeptide with a second amino acid sequence, wherein the first and second amino acid sequences do not occur naturally as part of a single polypeptide chain.

Accordingly, the invention provides a method, or an assay, for detecting, quantifying and/or monitoring the formation and/or stability of a multimeric complex, e.g., a ternary complex. The method includes providing a sample that includes at least three binding members under conditions that allow the formation of a multimeric complex to occur; detecting, quantifying and/or monitoring a change in the level of the multimeric complex (e.g., by detecting the formation and/or stability of the multimeric complex over a specified time interval, or in the presence of absence of a test agent).

For example, a binding member of the multimeric complex can be a peptide, a polypeptide (e.g., a cytokine, a chemokine, a growth factor and/or a receptor thereof), a large or small molecule (e.g., a macrolide or a polyketide), or any combination thereof. In one embodiment, the multimeric complex includes a first binding member, e.g., a receptor ligand (e.g., a cytokine); a second binding member, e.g., a ligand receptor (e.g., a cytokine receptor), and a third binding member, e.g., a ligand co-receptor (e.g., a cytokine receptor subunit that interacts with the cytokine receptor and/or the cytokine). For example, the multimeric complex can be a ternary complex that includes IL-13 as a first binding member, an IL-13 receptor α1 (IL-13Rα1) as a second binding member, and an IL-4 receptor (IL-4Rα) as a third binding member.

In a related aspect, a method, or assay, for identifying an agent that modulates, e.g., inhibits or increases, the formation and/or stability of a multimeric complex, e.g., a ternary complex, is disclosed. The method, or the assay, includes: contacting a sample that includes the first, second and third binding members with a test agent under conditions that allow the formation of the complex to occur; detecting the presence of the complex in the sample contacted with the test agent relative to a reference sample (e.g., a control sample not exposed to the test agent; a control sample exposed to known modulator, e.g., inhibitor, of the complex; and/or a control sample exposed to an excess amount of an unlabeled binding member of the complex). A change (e.g., an increase or a decrease) in the level of the complex in the presence of the test agent, relative to the level of the complex in the reference sample, indicates that said test agent affects (e.g., increases or decreases) the formation and/or stability of said complex. In some embodiments, test agents that decrease complex formation by, e.g., about 1.5, 2, 5, 10 fold or higher, relative to a reference sample are preferred. The methods and assays disclosed herein can be used to identify or test modulators of a signaling event, e.g., a cytokine signaling event. For example, test agents that modulate, e.g., inhibit, IL-13 signaling can be identified using the methods disclosed herein by identifying agents that (a) modulate, e.g., interfere with, the formation and/or stability of an IL-13 binary complex (e.g., by modulating, e.g., interfering with, an interaction between IL-13 and IL-13Rα1) and/or (b) by modulating, e.g., interfering with, the formation and/or stability of an IL-13 ternary complex (e.g., by interfering with the interaction between the binary complex and IL-4R).

In other embodiments, the method, or assay, further includes contacting the multimeric complex with a known inhibitor of the complex, or an excess amount of one or more of the binding members (e.g., an excess amount of unlabeled binding member) to detect the rate of dissociation of the complex. Such step can be carried out in the absence or presence of a test agent to detect the effect of the test compound on the inhibition/rate of dissociation of the complex. A change in binding (e.g., complex formation) and/or activity, in the presence or absence of the test agent, is indicative that the test agent modulates the dissociation of the complex, and/or modulates the interaction of the known inhibitor with the complex.

In other embodiments, the method, or assay, further includes the step(s) of comparing binding of the test agent to the complex compared to the binding of the known compound to the complex. The method, or assay, can additionally, optionally, include detecting the interaction (e.g., binding) of the test agent to a complex of two or more of the binding members, relative to the individual members.

Test agents can be, for example, a polypeptide (e.g., an antibody molecule, a soluble receptor), large or small molecule (e.g., a naturally occurring molecule or a synthetic molecule (e.g., a member of a combinatorial library). In one embodiment, the test agent interacts, e.g., binds to, at least one of the binding members of the multimeric complex. Test agents can be produced recombinantly, or as a natural product of bacteria, actinomycetes, yeast or other organisms; or produced chemically (e.g., small molecules, including peptidomimetics).

A variety of assay formats will suffice and, in light of the present disclosure, those not expressly described herein will nevertheless be understood by one of ordinary skill in the art. Assay formats which approximate such conditions as formation of protein complexes, enzymatic activity, and may be generated in many different forms, and include assays based on cell-free systems, e.g., purified proteins or cell lysates, as well as cell-based assays which utilize intact cells. Simple binding assays can be used to detect compounds that inhibit or potentiate the interaction between binding members of the complex, or the binding of the complex to a substrate.

In certain embodiments, the present invention provides a reconstituted preparation including one or more binding members. In one embodiment, all binding members of the complex are added simultaneously in a sample, e.g., a reaction mixture. In other embodiments, the sample is prepared by adding the binding members sequentially in any order, e.g., forming a mixture of the first member (e.g., a cytokine) with a second member (e.g., a cytokine receptor), and adding the third member (e.g., a cytokine co-receptor). In another embodiment, a mixture of the second member (e.g., a cytokine receptor) and the third member (e.g., a cytokine co-receptor) is formed, followed by addition of the first member (e.g., a cytokine). In yet other embodiments, a mixture of the first member (e.g., a cytokine) and the third member (e.g., a cytokine co-receptor) is formed, followed by addition of the second member (e.g., a cytokine receptor). Any order or combination of the binding members can be used.

Assays of the present invention include labeled in vitro protein-protein binding assays, immunoassays for protein binding, and the like, as described in more detail below. In one embodiment, the sample is a cell lysate or a reconstituted system (e.g., cell membrane or soluble components). The reconstituted complex can comprise a reconstituted mixture of at least semi-purified proteins. By semi-purified, it is meant that the proteins utilized in the reconstituted mixture have been previously separated from other cellular proteins. For instance, in contrast to cell lysates, proteins involved in the complex formation are present in the mixture to at least 50% purity relative to all other proteins in the mixture, and more preferably are present at 90-95% purity. In certain embodiments, the reconstituted protein mixture is derived by mixing highly purified proteins such that the reconstituted mixture substantially lacks other proteins (such as of cellular origin) which might interfere with or otherwise alter the ability to measure the complex assembly and/or disassembly. In certain embodiments, assaying in the presence and absence of a candidate compound, can be accomplished in any vessel suitable for containing the reactants. Examples include microtitre plates, test tubes, and micro-centrifuge tubes. Alternatively, the sample can include cells in culture, e.g., purified cultured or recombinant cells, or in vivo in an animal subject.

In certain embodiments, methods and assays can be developed which detect test agents on the basis of their ability to interfere with assembly, stability and/or function of a complex of the invention. Detection and quantification of the complex provide a means for determining the test agent's efficacy at inhibiting (or potentiating) interaction between the binding members. The efficacy of the test agent can be assessed, e.g., by generating and evaluating dose response or kinetics data obtained with the test agent. Moreover, a control assay can also be performed to provide a baseline for comparison. In one embodiment, the formation of complexes in the control assay is quantitated in the absence of the test compound.

In certain embodiments, the methods and assays of the invention detect a change in multimeric complex formation and/or stability by detecting one or more of: a change in the binding or physical formation of the complex itself, e.g., by biochemical detection, affinity based detection (e.g., Western blot, affinity columns), immunoprecipitation, fluorescence resonance energy transfer (FRET)-based assays (e.g., FRET or Time Resolved FRET assays (TR-FRET), surface plasmon resonance (SPR), spectrophotometric means (e.g., circular dichroism, absorbance, and other measurements of solution properties); a change, e.g., an increase or a decrease, in signal transduction, e.g., phosphorylation and/or transcriptional activity; a change, e.g., increase or decrease, cell function. In embodiments where the ternary complex includes IL-13 and IL-13 receptors, one or more of the following IL-13-associated activities can be evaluated: induction of CD23 expression; production of IgE by B cells; phosphorylation of a transcription factor, e.g., STAT protein (e.g., STAT6 protein); antigen-induced eosinophilia in vivo; antigen-induced bronchoconstriction in vivo; drug-induced airway hyperreactivity in vivo; eotoxin levels in vivo; and/or histamine release by basophils. In one embodiment, the test agent is identified and re-tested in the same or a different assay. For example, a test agent is identified in an in vitro or cell-free system, and re-tested in an animal model or a cell-based assay. Any order or combination of assays can be used. For example, a high throughput assay can be used in combination with an animal model or tissue culture.

In yet other embodiments, the methods and assays described herein may be used to identify previously unidentified components within a multimeric complex. The methods, or assays, include: (1) detectably identifying a library of candidate binding member (e.g., labeling a library of candidate members with a FRET donor); (2) detectably identifying at least one known member of the complex (e.g., labeling at least one known member of the complex with a FRET acceptor); (3) contacting said identified library with said identified at least one member of the complex, under conditions that allow an interaction to occur, wherein the interaction of the library member with the at least one member of the complex results in a detectable signal; (4) detecting the signal generated, e.g., by performing FRET or TR-FRET analysis. A change, e.g., an increase, in the signal generated upon association of the library member with the at least one member of the complex is indicative the association and/or complex formation. The method, or assays, can optionally include the step of identifying and/or obtaining the complex.

In embodiments where the methods and assays detect a change in multimeric complex formation and/or stability by FRET and/or TR-FRET, two or more of the binding members of the multimeric complex can be labeled with fluorescent molecules having the proper emission and excitation spectra, such that when brought into close proximity with one another can exhibit fluorescence resonance energy transfer. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. FRET-based assays are described in more detail herein.

In general, where the assay is a binding assay involving fluorescent emission (whether protein-protein binding, compound-protein binding), one or more of the binding members may be joined to a label. The label can be attached directly or indirectly to provide a detectable signal when brought to close proximity. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin. For the specific binding members, the complementary member would normally be labeled with a molecule that provides for detection, in accordance with known procedures.

Assays or detection methods can be used to identify test agents that modulate, e.g., interfere with, the formation and/or stability of a binary and/or the ternary IL-13 complex. For example, this method may be used to identify test agents that modulate, e.g., interfere with, an interaction between (a) IL-13 and IL-13Rα1, (b) IL-4Rα and IL-13Rα1, (c) IL-13 and IL-4R, as well as (c) test agents that modulate, e.g., interfere, with an interaction among IL-13, IL-13Rα1 and IL-4Rα, by modulating an interaction between two or more of these binding agents. Without being bound by theory, IL-13 is believed to interact initially with IL-13Rα1 forming an initial binary complex, which complex then interacts with IL-4Rα. Test agents that modulate, e.g., interfere with, one or more of these interactions can be evaluated using the methods and assays described herein. The assays and methods described herein may be adapted to detect formation and/or stability of other multimeric complexes, e.g., other ternary complexes, including but not limited to, for example, complexes of an interleukin and its receptors chosen from one of more of: interleukin 2 (IL-2), interleukin 4 (IL-4), interleukin 5 (IL-5), interleukin 6 (IL-6), interleukin 10 (IL-10), interleukin 15 (IL-15), interleukin 21 (IL-21), and/or interleukin 22 (IL-22).

In one exemplary embodiment where an IL-13 multimeric complex is evaluated, at least two of the binding members can be labeled for FRET detection. One of ordinary skill will appreciate that the methods and assays described herein can be practiced by labeling the at least two binding members with any combination of suitable FRET acceptor and donor. In one embodiment, the first and the third binding members (e.g., a IL-13 and IL-4Rα) are labeled for FRET detection, for example, by labeling IL-13 with a suitable FRET donor and IL-4Rα with a suitable FRET acceptor. For example, IL-13 may be labeled (e.g., directly labeled) with europium chelate (Eu) and IL-4Rα may be labeled (e.g., directly labeled) with Alexa Fluor 647 (FL647) or Cy5, using the methods described herein. In another embodiment, the second and third binding members (e.g., a IL-13 and IL-4Rα, respectively) may be labeled with a suitable FRET donor and acceptor. For example, IL-13Rα1 may be labeled (e.g., directly labeled) with europium chelate (Eu) and IL-4R may be labeled (e.g., directly labeled) with Alexa Fluor 647 (FL647) or Cy5, using the methods described herein. Such methods and assays may be used to identify test agents that interfere with the formation of a ternary complex. For example, these methods and assays may be used to identify test agents that interfere with the interaction between the binary complex of IL-13 and IL-13Rα1, and IL-4R. One of ordinary skill will appreciate that this method may also be practiced to achieve the same result by labeling IL-13Rα1 with a suitable FRET acceptor and IL-4R with a suitable FRET donor.

In some embodiments, the screening assays described herein, e.g., a TR-FRET assay, may be performed in vitro using isolated binding members. In such a system, each component of the screen may be added separately in wells of a multi-well plate, for example 96, 384, and 1536-well plates. In some embodiments, the multimeric complex will be allowed to form prior to the addition of the test agent to be screened. In other embodiments, the members of the complex and the test agent will be added together, i.e., at the same time or simultaneously, with one or more of the members of the complex. In some embodiments, the screening assay evaluates a plurality of different test agents, at a fixed or a range of concentrations. In some embodiments, the screening assay will screen a known or previously identified inhibitor of the complex.

In some embodiments, the methods and assays described herein may be performed using TR-FRET. In such a system a detected decrease in the TR-FRET signal, e.g., a 0.5%, 1%, 1.5%, 3%, 5%, 10%, 20%, or higher is indicative that a test agent is an inhibitor of the complex. In some embodiments, the percent decrease will be compared to a previously established percent decrease for the same molecule, for example, when validating a molecule. In some embodiments, a threshold percent decrease will be established prior to the screen. Test agents that meet said threshold value are considered to be considered effective.

In other embodiments, the methods and assays described herein may be performed in vivo, using for example Bioluminescence Resonance Energy Transfer (BRET). In such a system, the members of the multimeric complex may be overexpressed as fusion proteins within a cell. The fusion may be a detectable label, e.g., a fluorophore selected from Table 2, and at least two of the members of the complex will be labeled. In some embodiments, the complex may be labeled indirectly using, for example labeled antibodies. In such a system, the components of the ternary complex may be overexpressed proteins, e.g., fusion proteins containing a detectable marker, e.g., a six histidine tag or an Xpress™ epitope, that can be detected (i.e., probed) with a commercially available antibody. In some embodiments, the components of the ternary complex may be endogenous proteins that are probed with at least two protein specific antibodies with labels that are capable of BRET. In such a system a detected decrease in the BRET signal, e.g., a 0.5%, 1%, 1.5%, 3%, 5%, 10%, 20%, and above will be considered a positive indication that a screened molecule is an inhibitor of a ternary complex.

In other embodiments, the method, or assay, includes providing a step based on proximity-dependent signal generation, e.g., a two- or three-hybrid assay that includes a first binding member (e.g., a cytokine), a fusion protein (e.g., a fusion protein comprising a portion of the second binding member (e.g., a cytokine receptor)), and another fusion protein (e.g., a fusion protein comprising a portion of the third binding member (e.g., a cytokine co-receptor), using cells in culture, e.g., purified cultured or recombinant cells. The method, or assay includes: contacting the two- or three-hybrid assay with a test agent, under conditions wherein said hybrid assay detects a change in the formation and/or stability of the complex, e.g., the formation of the complex initiates transcription activation of a reporter gene. Examples of two- or three-binding assays are described in Licitra, E. et al. (1996) Proc. Natl. Acad. Sci. 93: 12817-12821; U.S. Pat. No. 5,283,317; WO94/10300; Zervos et al. (1993) Cell 72: 223-232; Madura et al. (1993) J. Biol. Chem. 268: 12046-12054; Bartel et al. (1993) Biotechniques 14: 920-924; and Iwabuchi et al. (1993) Oncogene 8: 1693-1696, the contents of all of which are incorporated by reference.

A variety of other reagents may be included in the assays and methods of the invention. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial compounds may be used. The mixture of components is added in any order that provides for the requisite binding. Incubations are performed at any suitable temperature, typically between 4 and 40° C. Incubation periods are selected for optimum activity, but may also be optimized to facilitate rapid high-throughput screening.

In certain embodiments, association between any two polypeptides in a complex or between the complex and a substrate polypeptide, may be detected by a variety of techniques, many of which are described more extensively herein. For instance, modulation in the formation of complexes can be quantified using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction.

In certain embodiments, one of the binding members of a complex can be immobilized to facilitate separation of the complex from uncomplexed forms of one of the polypeptides, as well as to accommodate automation of the assay. Affinity matrices or beads are described herein that contain the ligand (or other components of the complex) that permits other components of the complex to be bound to an insoluble matrix. Test compound are incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

In many screening assays which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test agent. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

Some of the detection techniques used in the assays and methods of the invention are described in more detail herein.

### Fluorescence Resonance Energy Transfer (FRET)

In some embodiments, the methods described herein use FRET-based homogenous assays for detection of the multimeric complexes. FRET-based assays are described in U.S. Pat. No. 5,981,200, which is herein incorporated by reference. FRET requires at least two dye molecules: a first dye that serves as a FRET donor and a second dye that serves as a FRET acceptor. Typically, a FRET donor is an energy donor and a FRET acceptor is an energy acceptor. FRET is the energy transfer that takes place between the FRET donor and the FRET acceptor, as described in more detail below, and is the signal that is measured during a so-called FRET assay.

Fluorescent molecules having the proper emission and excitation spectra that are brought into close proximity with one another can exhibit FRET. FRET is the transfer of energy from a FRET donor to a FRET acceptor. This process occurs as follows: First, a FRET donor is excited, for example, using a picosecond laser pulse, and is converted, by absorption of energy in the form of a photon, from a ground state into an excited state. Second, the FRET donor emits this newly absorbed energy as fluorescent light. Third, if the excited donor molecule is close enough to a suitable acceptor molecule, the excited state can be transferred from the donor to the acceptor in the form of fluorescent light. This energy transfer is known as FRET. Fourth, FRET results in a decrease in the fluorescence or luminescence of the donor and, if the acceptor is itself luminescent, results in an increased luminescence of the acceptor. The light emitted by the acceptor can be measured using a FRET-detection system, and is proportional to the FRET. Thus, the information gathered can be used for qualitative and quantitative analysis. In some embodiments, the light emitted from the donor will be a of a different wavelength than the light emitted from the acceptor.

The efficiency of FRET, i.e., the signal produced when energy is transferred from the donor to the acceptor dye is dependent on the distance (1/d) between the donor and acceptor dye and FRET only occurs efficiently when the donor and acceptor are very close together. The decrease in signal depends on the sixth power of the separation distance. Thus, FRET measures distance dependent interactions. Measurements made using FRET are on the scale of about 15-100 Å.

Thus, as used herein, interaction means changes in the distance between biomolecules that can be detected by FRET measurement. In order to detect this interaction, it is necessary that a FRET donor as well as a FRET acceptor are coupled to one or more biomolecules and that the interaction between these one or more biomolecules leads to a change in the distance between the FRET donor and the FRET acceptor.

In some embodiments, FRET may include, but is not limited to; (A) the FRET donor and the FRET acceptor bound to different molecules in a binding pair; (B) the FRET donor and the FRET acceptor bound to different regions within a single molecule; and (C) the FRET donor and the FRET acceptor bound to two different molecules in a ternary complex. However, in (C), the two separate molecules that the FRET donor and the FRET acceptor are attached to must complex in such a way that efficient energy transfer can occur between the donor and the acceptor.

Thus, FRET can be manifested as (A) a reduction in the intensity of the fluorescent signal from the FRET donor; (B) a reduction in the lifetime of the excited state of the FRET donor; and/or (C) re-emission of fluorescent light typically at the longer wavelengths (lower energies) characteristic of the acceptor.

Energy acceptors can either be selected such that they suppress the energy released by the donor, which are referred to as quenchers, or the fluorescence resonance energy acceptors can themselves release fluorescent energy, i.e., they fluoresce. Such energy acceptors are referred to as fluorophore groups or as fluorophores. Metallic complexes are suitable as fluorescence energy donors as well as fluorescence energy acceptors. Fluorophores chosen for use in FRET are generally bright and occur on a timescale ranging from 10⁻⁹ seconds to 10⁻⁴ seconds. Such brightness and timescale facilitate the detection of FRET and allow the use of a variety of detection methods.

In some embodiments, the FRET donor and the FRET acceptor are chosen based on one or more, including all, of the following: (1) the emission spectrum of the FRET donor should overlap with the excitation spectrum of the FRET acceptor; (2) The emission spectra of the FRET partners (i.e., the FRET donor and the FRET acceptor) should show non-overlapping fluorescence; (3) the FRET quantum yield (i.e., the energy transferred from the FRET donor to the FRET acceptor) should be as high as possible (for example, FRET should have about a 1-100%, e.g., a 30%, 40%, 50%, 60%, 70%, 80%, 85%, 90%, 95%, 98%, and 99% efficiency over a measured distance of 1-20 nm, e.g., 5-10 nm); (4) the FRET signal (i.e., fluorescence) must be distinguishable from fluorescence produced by the sample, e.g., autofluorescence; and (5) the FRET donor and the FRET acceptor should have half lives that facilitate detection of the FRET signal (e.g., FRET can be bright and can occur on a timescale ranging from 10−⁹ seconds to 10−⁴ seconds, as described above).

In some embodiments, the FRET donor and the FRET acceptor may be chosen based upon one or more of the fluorophores listed in Table 2.

The following information may also be considered when selecting a FRET donor and FRET acceptor combination.

U.S. Pat. No. 5,998,146, herein incorporated by reference, describes the use of lanthanide chelate complexes, in particular of europium and terbium complexes combined with fluorophores or quenchers. It also underscores the advantageous properties of the long-lived lanthanide chelate complexes.

FRET systems based on metallic complexes as energy donors and dyes from the class of phycobiliproteins as energy acceptors are known in the prior art (EP 76 695; Hemmilae, Chemical Analysis 117, John Wiley & Sons, Inc., (1991) 135-139). Established commercial systems (e.g. from Wallac, OY or C is Bio Packard) use a FRET pair consisting of a lanthanide chelate as the metallic complex and a phycobiliprotein.

The advantageous properties of the lanthanide-chelate complexes in particular of europium or terbium complexes are known and can be used in combination with quenchers as well as in combination with fluorophores.

Ruthenium complexes per se are used as fluorophores or luminophores especially for electro-chemoluminescence. Ruthenium-chelate complexes are, for example, known from EP 178 450 and EP 772 616 in which methods for coupling these complexes to biomolecules are also described. Their use as energy donors in FRET systems is not discussed there.

Allophycocyanins have excellent properties such as unusually high extinction coefficients (about 700 000 L/M cm) and also extremely high emission coefficients. These are ideal prerequisites for their use as fluorophore acceptors in FRET systems. Moreover these dyes are known to be readily soluble in water and stable.

The term low molecular fluorophore refers to fluorophoric dyes having a molecular weight between 300 and 3000 Da. Such low molecular fluorophoric groups such as xanthenes, cyanins, rhodamines and oxazines have considerable disadvantages compared to the APCs with regard to important characteristics. Thus for example their extinction coefficients are substantially lower and are in the range of ca. 100 000 L/M cm. It is also known that unspecific binding due to the hydrophobic properties of these chromophores is a potential disadvantage for these dyes as acceptors in FRET systems.

Methods for labeling a molecule for FRET are described in the appended examples and are known in the art. For example, binding members can be labeled directly or indirectly (e.g., via a tag or using avidin-streptavidin interactions), as described by Yang et al. (2006) Analytical Biochemistry, 351:158-160, which is herein incorporated by reference. In some embodiments, binding members can be labeled directly. Methods for directly labeling a binding member, e.g., a protein, are disclosed in the appended Examples and are known in the art. These methods include labeling the molecules with a FRET donor and a FRET acceptor. Generally binding members, e.g., proteins, may be prepared in a 100 μM bicarbonate buffer (pH 8.3), to a final protein concentration of about 1.0 mg/ml. This solution may then be mixed with a desired label, and incubated at room temperature for about one hour. Unincorporated label can then be separated from the molecule, e.g., the protein, using a micro column.

### Time Resolved FRET (TR-FRET)

In some embodiments, the methods and assays of the invention make use of homogeneous TR-FRET assay techniques. TR-FRET is a combination of time-resolved fluorescence (TRF) and FRET. TRF reduces background fluorescence by delaying reading the fluorescent signal, for example, by about 10 nano seconds. Following this delay (i.e., the gating period), the longer lasting fluorescence in the sample is measured. Thus, using TR-FRET, interfering background fluorescence, that may for example be due to interfering substances in the sample, is not co-detected, but rather, only the fluorescence generated or suppressed by the energy transfer is measured. The resulting fluorescence of the TR-FRET system is determined by means of appropriate measuring devices. Such time-resolved detection systems use, for example, pulsed laser diodes, light emitting diodes (LEDs) or pulsed dye lasers as the excitation light source. The measurement occurs after an appropriate time delay, i.e. after the interfering background signals have decayed. Devices and methods for determining time-resolved FRET signals are described in the art.

This technique requires that the signal of interest must correspond to a compound with a long fluorescent lifetime. Such long-lived fluorescent compounds are the rare earth lanthanides. For example, Eu³⁺ has a fluorescent lifetime in the order of milliseconds.

TR-FRET requires a FRET donor and a FRET acceptor, as described above. As with FRET, a TR-FRET donor and acceptor pair can be selected based on one or more, including all, of the following: (1) the emission spectrum of the FRET donor should overlap with the excitation spectrum of the FRET acceptor; (2) The emission spectra of the FRET partners (i.e., the FRET donor and the FRET acceptor) should show non-overlapping fluorescence; (3) the FRET quantum yield (i.e., the energy transferred from the FRET donor to the FRET acceptor) should be as high as possible (for example, FRET should have about a 1-100%, e.g., a 30%, 40%, 50%, 60%, 70%, 80%, 85%, 90%, 95%, 98%, and 99% efficiency over a measured distance, of 1-20 nm, e.g., 5-10 nm); (4) the FRET signal (i.e., fluorescence) must be distinguishable from fluorescence produced by the sample, e.g., autofluorescence; and (5) the FRET donor and the FRET acceptor should have half lives that allow detection of the FRET signal (e.g., FRET can be bright and can occur on a timescale ranging from 10−⁹ seconds to 10−⁴ seconds).

In some embodiments, the TR-FRET donor and the TR-FRET acceptor may be chosen based upon one or more of the fluorophores listed in Table 2.

In some embodiments, the TR-FRET donor may be a lanthanide. In some embodiments, the lanthanide may be europium (Eu), terbium (Tb), and samarium, including second generation and functional homologues of Eu, Tb, and samarium. As used herein, Eu includes Eu and all Eu homologues, e.g., Eu³⁺. In some embodiments, the TR-FRET donor may be DsRed. In some embodiments, the TR-FRET donor may be Ri2. It is to be understood that selection of the appropriate TR-FRET donor requires consideration of the above listed criteria and the specific TR-FRET acceptor selected.

In some embodiments, the TR-FRET acceptor may be selected from the group consisting of fluorescein, Cy5, allophycocyanin (APC— e.g., XL665, d2, and BG-647), and fluorescent protein (e.g., GFP, CFP, YFP, BFP, and RFP).

In some embodiments, the TR-FRET donor may be terbium and the TR-FRET acceptor may be fluorescein. In some embodiments, the TR-FRET donor may be Eu and the TR-FRET acceptor may be Cy5 or APC (e.g., XL665, d2, and BG-647).

In some embodiments, the TR-FRET donor and the TR-FRET acceptor may be combined with a second compound that enhances the function of the TR-FRET donor and/or the TR-FRET acceptor. For example, the TR-FRET donor and the TR-FRET acceptor may be combined with cryptate encapsulation to extend the half-life of the fluorophore. Alternatively, or in addition, the TR-FRET donor the TR-FRET acceptor may be combined with, e.g., DELFIA® enhancement system. In some embodiments, the TR-FRET donor and the TR-FRET acceptor may be combined with, for example buffers, salts, enhancers, chelators, and stabilizers (e.g., photo-stabilizers) that enhance or extend the life or detection of the TR-FRET signal.

A variety of other reagents may also be included in the screening assays described above. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial compounds, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening.

Molecules, e.g., proteins, may be labeled directly or indirectly with suitable TF-FRET donors and acceptors, as described above.

In some embodiments, one or more combinations of the above described assays may be performed. For example, TR-FRET may be performed with a heterogeneous assay, e.g., surface plasmon resonance (SPR) or ELISA.

### Surface Plasmon Resonance

In some embodiments, the methods described herein include methods for screening for inhibitors of a ternary complex using heterogeneous assay techniques.

An exemplary heterogeneous screening assay is surface plasmon resonance (SPR). SPR is a phenomenon that occurs when light is reflected off thin metal films. SPR measures biomolecular interactions in real-time in a label free environment. SPR is performed by immobilizing at least one molecule to the sensor surface while the other one or more molecules is free in solution and passed over the sensor surface. In some embodiments, two or more molecules may be attached to the sensor surface. In some embodiments, the two or more molecules are independent molecules and do not interact. In some embodiments, the two or more molecules may interact form a complex, for example, a binary complex. In some embodiments, the two or more molecules may form, e.g., a ternary complex, a quaternary complex, or a quinary complex. In some embodiments, a complex will be formed before immobilization to the sensor surface. Measurements, e.g., association and dissociation, are generally recorded in arbitrary units and are displayed graphically. SPR is not limited to proteins. Interactions between DNA-DNA, DNA-protein, lipid-protein and hybrid systems of biomolecules and non-biological surfaces can be investigated.

SPR is routinely performed using an SPR-machine. The most common SPR-machine is known commercially as Biacore, and is currently marketed by GE Healthcare. Other SPR systems include, but are not limited to Nanofilm Surface analysis (Nanofilm Technology, Germany), BI Biosensing Instrument (Biosensing Instrument Inc., USA). SPR sensor chips are available commercially through Bio-Rad (USA).

Methods for immobilizing molecules, including proteins on the surface of a chip are known in the art. In some embodiments, methods include, for example, surfaces provided by chips (e.g., research grade CM5 sensor chip). Chips may be activated using a 30 second pulse of N-ethyl-N-(2-dimethylaminopropyl) carbodiimide hydrochloride mixed with N-hydroxylsuccinimide (NHS-EDC). Molecules, e.g., proteins, suspended in 10 mM sodium acetate pH 4.0 may then be injected over the activated surfaces for one to two minutes to achieve the desired surface densities. Surfaces may then be deactivated using a 5-minute injection of 1 M ethanolamine-HCl prior to performing kinetic experiments.

### High Throughput Screening Assays

In some embodiments, the methods described herein include High throughput screening (HTS) methods.

HTS is a relative term, but is generally defined as the testing of 10,000 to 100,000 compounds per day, accomplished with mechanization that ranges from manually operated workstations to fully automated robotic systems.

HTS screening techniques generally provide advantages over non-HTS methods as they are faster, due to automation, highly reproducible, and cost effective. HTS allows large numbers of samples, e.g., inhibitors of a ternary complex, to be screened and/or validated per day. HTS can considerably educe the cost of drug discovery and quality control.

In some embodiments, HTS may be performed using 96, 384, and 1536-well microtiter plates. In some embodiments, FRET and or TR-FRET may be used in a high throughput system to identify or verify inhibitors of a ternary complex.

### Kits

The present invention also includes kits. In some embodiments, the kits comprise one or more labeled molecules of a multimeric complex. The type of molecules and labels may vary depending on the requirements of the screen for which a particular kit is being supplied.

In some embodiments, a kit may contain one or more of the following in a package or container: (1) a first molecule; (2) a second molecule; (3) a third molecule; (4) a first label; (5) a second label; (6) a suitable solution comprising one or more agents to facilitate the formation of a ternary complex; (7) one or more agents to promote detection of the first and second label, including a third signal generated by a combination of the first and the second label; and (8) instructions for use. Embodiments in which two or more, including all, of the components (1)-(8), are found in the same container can also be used.

When a kit is supplied, the different components of the compositions included can be packed in separate containers and admixed immediately before use. If the components will remain stable after admixing, the components may be admixed at a time before use other than immediately before use, including, for example, minutes, hours, days, months and years, before use, and at the time of manufacture.

The compositions included in particular kits of the present invention can be supplied in containers of any sort such that the life of the different components are optimally preserved and are not adsorbed or altered by the materials of the container. Suitable materials for these containers may include, for example, glass, organic polymers (e.g., polycarbonate and polystyrene), ceramic, metal (e.g., aluminum), an alloy, or any other material typically employed to hold similar reagents. Exemplary containers may include, without limitation, test tubes, vials, flasks, bottles, syringes, and the like.

As stated above, the kits can also be supplied with instructional materials. These instructions may be printed and/or may be supplied, without limitation, as an electronic-readable medium, such as a floppy disc, a CD-ROM, a DVD, a zip disc, a video cassette, an audiotape, and a flash memory drive. Alternatively, the instructions may be published on an internet web site or may be distributed to the user as an electronic mail.

The above described kits include kits prepared to screen for modulators, e.g., inhibitors, of the interactions within a ternary complex, such as, ternary complexes of IL-13, IL-2, IL-6, IL-4, IL-5, IL-10, IL-15, IL-21, and IL-22.

### Multimeric Complexes

The assays and methods described herein may be adapted to detect formation and/or stability of multimeric complexes, e.g., ternary complexes, including but not limited to, for example, complexes of an interleukin and its receptors chosen from one of more of: IL-13, IL-2, IL-6, IL-4, IL-5, IL-10, IL-15, IL-21 and/or IL-22. Some of these complexes are described in more detail herein.

### IL-13 and IL-4

Interleukin-13 (IL-13) is a previously characterized cytokine secreted by T lymphocytes and mast cells (McKenzie et al. (1993) Proc. Natl. Acad. Sci. USA 90:3735-39; Bost et al. (1996) Immunology 87:663-41). The term “IL-13” refers to interleukin-13, including full-length unprocessed precursor form of IL-13, as well as the mature forms resulting from post-translational cleavage. The term also refers to any fragments and variants of IL-13 that maintain at least some biological activities associated with mature IL-13, including sequences that have been modified. The term “IL-13” includes human IL-13, as well as other vertebrate species. Several pending applications disclose antibodies against human and monkey IL-13, IL-13 peptides, vectors and host cells producing the same, for example, U.S. Application Publication Nos. 2006/0063228A and 2006/0073148. The contents of all of these publications are incorporated by reference herein in their entirety. Inhibition of IL-13 in various animal models of asthma results in attenuated disease (Grunig et al., Science, 282:2261-2263, 1998; Wills-Karp et al., Science, 282:2258-2261, 1998; Bree et al., Clin. Immunol., 119:1251-1257, 2007).

IL-4 has two signaling receptor complexes. For each receptor, IL-4 first binds to IL-4R with high affinity, and this binary complex then binds to either the γc chain or the IL-13Rα1 chain to initiate signaling (Aversa et al., J. Exp. Med., 178:2213-2218, 1993; Zurawski et al., Ann. Rev. Immunol., 21:425-456, 2003). Some differences between IL-4 and IL-13 activity can be attributed to IL-4 interaction with the IL-4R-γc complex. The γc chain, which is utilized by IL-4 but not IL-13, is expressed mainly by T cells and other hematopoietic cells, whereas IL-13Rα1, utilized by both IL-4 and IL-13, is expressed by non hematopoietic cells (Wynn et al., Ann. Rev. Immunol., 21:425-456, 2003). However, differences between IL-4 and IL-13 biological functions occur, even on non-hematopoietic cells that express the identical receptor components for both cytokines. Mice that over express either IL-13 or IL-4 in the bronchial epithelium, both have goblet-cell metaplasia and lung inflammation, but only the IL-13 overexpressing mice have subepithelial fibrosis and smooth muscle cell proliferation, associated with airway hyperresponsiveness (Zhu et al., J. Clin. Invest., 103:779-88, 1999; Rankin et al., Proc. Natl. Acad. Sci., 93:7821-5, 1996).

Formation of the IL-13 ternary complex involves a sequential series of steps. IL-13 initially binds to the IL-13 receptor (IL-13Rα1) with low affinity (2-10 nM), and forms an IL-13 binary complex. This binary complex lacks signaling activity (Aman et al., J. Biol. Chem., 271:29265-70, 1996; Hilton et al., Proc. Natl. Acad. Sci., 93:497-501, 1996; Caput et al., J. Biol. Chem., 271:16921-6, 1996; Miloux et al., FEBS Lett., 401:163-6, 1997). The binary IL-13/IL-13Rα1 complex then binds to the alpha chain of the IL-4 receptor (IL-4R), resulting in the formation of the IL-13 ternary complex. This ternary complex is the functional IL-13 complex, which serves as a high affinity receptor that mediates STAT6 phosphorylation and downstream cellular responses (Caput et al., J. Biol. Chem., 271:16921-6, 1996; Miloux et al., FEBS Lett., 401:163-6, 1997).

In addition, several polymorphisms have been identified in the IL-13 locus on chromosome 5q31 (Graves et al., Journal of Allergy and Clinical Immunology, 105:506-513, 2000; Pantelidis et al., Genes Immunol., 1:341-5, 2000). One of these, G2004A, produces a variant in the coding region of the gene and an amino acid change at position 110 of a nonconservative substitution from arginine to glutamine (R110Q). There is a strong association with this variant and elevated IgE levels, atopic dermatitis, rhinitis, and asthma (Graves et al., Journal of Allergy and Clinical Immunology, 105:506-513, 2000: Liu et al., Journal of Allergy and Clinical Immunology, 106:167-170, 2000; Heinzmann et al., Hum. Mol. Genet., 9:549-559, 2000).

Accordingly, inhibition of IL-13 and/or IL-4 can be useful in ameliorating the pathology of a number of inflammatory and/or allergic conditions, including, but not limited to, respiratory disorders, e.g., asthma; chronic obstructive pulmonary disease (COPD); other conditions involving airway inflammation, eosinophilia, fibrosis and excess mucus production, e.g., cystic fibrosis and pulmonary fibrosis; atopic disorders, e.g., atopic dermatitis, urticaria, eczema, allergic rhinitis; inflammatory and/or autoimmune conditions of, the skin (e.g., atopic dermatitis), gastrointestinal organs (e.g., inflammatory bowel diseases (IBD), such as ulcerative colitis and/or Crohn's disease), liver (e.g., cirrhosis, hepatocellular carcinoma); scleroderma; tumors or cancers (e.g., soft tissue or solid tumors), such as leukemia, glioblastoma, and lymphoma, e.g., Hodgkin's lymphoma; viral infections (e.g., from HTLV-1); fibrosis of other organs, e.g., fibrosis of the liver, (e.g., fibrosis caused by a hepatitis B and/or C virus).

### IL-22

Interleukin-22 (IL-22) is a previously characterized class II cytokine that shows sequence homology to IL-10. Its expression is up-regulated in T cells by IL-9 or ConA (Dumoutier L. et al. (2000) Proc Natl Acad Sci USA 97 (18):10144-9). Studies have shown that expression of IL-22 mRNA is induced in vivo in response to LPS administration, and that IL-22 modulates parameters indicative of an acute phase response (Dumoutier L. et al. (2000) supra; Pittman D. et al. (2001) Genes and Immunity 2:172), and that a reduction of IL-22 activity by using a neutralizing anti-IL-22 antibody ameliorates inflammatory symptoms in a mouse collagen-induced arthritis (CIA) model. Thus, IL-22 antagonists, e.g., neutralizing anti-IL-22 antibodies and fragments thereof, can be used to induce immune suppression in vivo, for examples, for treating autoimmune disorders (e.g., arthritic disorders such as rheumatoid arthritis); respiratory disorders (e.g., asthma, chronic obstructive pulmonary disease (COPD)); inflammatory conditions of, e.g., the skin (e.g., psoriasis), cardiovascular system (e.g., atherosclerosis), nervous system (e.g., Alzheimer's disease), kidneys (e.g., nephritis), liver (e.g., hepatitis) and pancreas (e.g., pancreatitis).

The term “IL-22” refers to interleukin-22, including full-length unprocessed precursor form of IL-22, as well as the mature forms resulting from post-translational cleavage. The term also refers to any fragments and variants of IL-22 that maintain at least some biological activities associated with mature IL-22, including sequences that have been modified. The term “IL-22” includes human IL-22, as well as other vertebrate species. The amino acid and nucleotide sequences of human and rodent IL-22, as well as antibodies against IL-22 are disclosed in, for example, U.S. Application Publication Nos. 2005-0042220 and 2005-0158760, and U.S. Pat. No. 6,939,545. The contents of all of these publications are incorporated by reference herein in their entirety.

IL-22 binds to a receptor complex consisting of IL-22R and IL-10R2, two members of the type II cytokine receptor family (CRF2) (Xie M. H. et al. (2000) J Biol Chem 275 (40):31335-9; Kotenko S. V. et al. (2001) J Biol Chem 276 (4):2725-32). Both chains of the IL-22 receptor are expressed constitutively in a number of organs. Epithelial cell lines derived form these organs are responsive to IL-22 in vitro (Kotenko S. V. (2002) Cytokine & Growth Factor Reviews 13 (3):22340). IL-22 induces activation of the JAK/STAT3 and ERK pathways, as well as intermediates of other MAPK pathways (Dumoutier L. et al. (2000) supra; Xie M. H. et al. (2000) supra; Dumoutier L. et al. (2000) J Immunol 164 (4):1814-9; Kotenko S. V. et al. (2001) J Biol Chem 276 (4):2725-32; Lejeune, D. et al. (2002) J Biol Chem 277 (37):33676-82

### IL-21

Human IL-21 is cytokine about a 131-amino acids in length that shows sequence homology to IL-2, IL-4 and IL-15 (Parrish-Novak et al. (2000) Nature 408:57-63). Despite low sequence homology among interleukin cytokines, cytokines share a common fold into a “four-helix-bundle” structure that is representative of the family. Most cytokines bind either the class I or the class II cytokine receptors. Class II cytokine receptors include the receptors for IL-10 and the interferons, whereas class I cytokine receptors include the receptors for IL2-IL7, IL-9, IL-11-13, and IL-15, as well as hematopoietic growth factors, leptin and growth hormone (Cosman, D. (1993) Cytokine 5:95-106).

Human IL-21R is a class I cytokine receptor that is expressed in lymphoid tissues, in particular by NK, B and T cells (Parrish-Novak et al. (2000) supra). The nucleotide and amino acid sequences encoding human interleukin-21 (IL-21) and its receptor (IL-21R) are described in WO 00/53761; WO 01/85792; Parrish-Novak et al. (2000) supra; Ozaki et al. (2000) Proc. Natl. Acad. Sci. USA 97:11439-114444. IL-21R has the highest sequence homology to IL-2 receptor beta chain and IL-4 receptor alpha chain (Ozaki et al. (2000) supra). Upon ligand binding, IL-21R associates with the common gamma cytokine receptor chain (gamma c) that is shared by receptors for IL-2, IL-3, IL-4, IL-7, IL-9, IL-13 and IL-15 (Ozaki et al. (2000) supra; Asao et al. (2001) J. Immunol. 167:1-5). The widespread lymphoid distribution of IL-21R suggests that IL-21 may play a role in immune regulation. Indeed, in vitro studies have shown that IL-21 significantly modulates the function of B cells, CD4.sup.+ and CD8.sup.+ T cells, and NK cells (Parrish-Novak et al. (2000) supra; Kasaian, M. T. et al. (2002) Immunity 16:559-569).

The term “IL21” refers to interleukin-21, including full-length unprocessed precursor form of IL-21, as well as the mature forms resulting from post-translational cleavage. The term also refers to any fragments and variants of IL-21 that maintain at least some biological activities associated with mature IL-21, including sequences that have been modified. The term “IL-21” includes human IL-21, as well as other vertebrate species.

### Test Agents

### Antibody Molecules

Antibody molecules provide an example of a test agent that can be evaluated practicing the methods and assays of the invention. Antibody molecules can be generated against the multimeric complexes disclosed herein that recognize one or more of the binding members of the complexes described herein in complexed and/or uncomplexed form.

As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites. Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a shark antibody. These antibody fragments are obtained using conventional techniques known to those with skill in the art, and the fragments are screened for utility in the same manner as are intact antibodies.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Generally, unless specifically indicated, the following definitions are used: AbM definition of CDR1 of the heavy chain variable domain and Kabat definitions for the other CDRs. In addition, embodiments of the invention described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to a protein target, or an epitope thereof. With respect to proteins (or protein mimetics), the antigen-binding site typically includes one or more loops (of at least four amino acids or amino acid mimics) that form an interface that binds to the protein target. Typically, the antigen-binding site of an antibody molecule includes at least one or two CDRs, or more typically at least three, four, five or six CDRs.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see, e.g., Saleh et al., Cancer Immunol. Immunother., 32:180-190 (1990)) and also because of potential allergic reactions (see, e.g., LoBuglio et al., Hybridoma, 5:5117-5123 (1986)).

Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG₁, IgG₂, IgG₃, IgG₄, IgA₁, and IgA₂. Each light chain includes an N-terminal variable (V) domain (VL) and a constant (C) domain (CL). Each heavy chain includes an N-terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is typically the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit the binds to the antigen, i.e., the antigen-binding fragment, or, e.g., the portion of the CH subunit that binds to and/or activates, e.g., an Fc receptor and/or complement. The CDRs typically refer to the Kabat CDRs, as described in Sequences of Proteins of Immunological Interest, US Department of Health and Human Services (1991), eds. Kabat et al. Another standard for characterizing the antigen binding site is to refer to the hypervariable loops as described by Chothia. See, e.g., Chothia, D. et al. (1992) J. Mol. Biol. 227:799-817; and Tomlinson et al. (1995) EMBO J. 14:4628-4638. Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Embodiments described with respect to Kabat CDRs can alternatively be implemented using similar described relationships with respect to Chothia hypervariable loops or to the AbM-defined loops.

Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992).

Antibody molecules can also include single domain antibodies. Single domain antibodies can include antibody molecules whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the invention, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies dervied from a variable region of NAR (“IgNARs”) are described in WO 03/014161 and Streltsov (2005) Protein Sci. 14:2901-2909. A single domain antibody is a naturally occurring single domain antibody known as heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs can be assayed using the methods of the present invention.

Numerous methods known to those skilled in the art are available for obtaining antibodies. For example, monoclonal antibodies may be produced by generation of hybridomas in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

In another embodiment, a monoclonal antibody is obtained from the non-human animal, and then modified, e.g., humanized, deimmunized, chimeric, may be produced using recombinant DNA techniques known in the art. A variety of approaches for making chimeric antibodies have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982), and may be made according to the teachings of PCT Publication WO92/06193 or EP 0239400).

An antibody may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

In certain embodiments, an antibody can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, Annu. Rev. Immunol 9:457-92, 1991; Capel et al., Immunomethods 4:25-34, 1994; and de Haas et al., J. Lab. Clin. Med. 126:330-41, 1995).

### Soluble Receptors and Receptor Fusions

Another example of a test agent that can be evaluated practicing the methods and assays of the invention are soluble receptors or fragments thereof. Examples of soluble receptors include the extracellular domain of a receptor, such as soluble tumor necrosis factor alpha and beta receptors (TNFR-1; EP 417,563 published Mar. 20, 1991; TNFR-2, EP 417,014 published Mar. 20, 1991; and reviewed in Naismith and Sprang, J. Inflamm. 47 (1-2):1-7, 1995-96, each of which is incorporated herein by reference in its entirety). In other embodiments, the soluble receptor includes the extracellular domain of interleukin-21 receptor (IL-21R) as described in, for example, US 2003-0108549 (the contents of which are also incorporated by reference).

The fusion protein can include a targeting moiety, e.g., a soluble receptor fragment or a ligand, and an immunoglobulin chain, an Fc fragment, a heavy chain constant regions of the various isotypes, including: IgG1, IgG2, IgG3, IgG4, IgM, IgA1, IgA2, IgD, and IgE. For example, the fusion protein can include the extracellular domain of a receptor, and, e.g., fused to, a human immunoglobulin Fc chain (e.g., human IgG, e.g., human IgG1 or human IgG4, or a mutated form thereof). In one embodiment, the human Fc sequence has been mutated at one or more amino acids, e.g., mutated at residues 254 and 257 from the wild type sequence to reduce Fc receptor binding. The fusion proteins may additionally include a linker sequence joining the first moiety to the second moiety, e.g., the immunoglobulin fragment. For example, the fusion protein can include a peptide linker, e.g., a peptide linker of about 4 to 20, more preferably, 5 to 10, amino acids in length; the peptide linker is 8 amino acids in length. For example, the fusion protein can include a peptide linker having the formula (Ser-Gly-Gly-Gly-Gly)y wherein y is 1, 2, 3, 4, 5, 6, 7, or 8. In other embodiments, additional amino acid sequences can be added to the N- or C-terminus of the fusion protein to facilitate expression, steric flexibility, detection and/or isolation or purification.

A chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) Current Protocols in Molecular Biology, John Wiley & Sons, 1992). Moreover, many expression vectors are commercially available that encode a fusion moiety (e.g., an Fc region of an immunoglobulin heavy chain). Immunoglobulin fusion polypeptides are known in the art and are described in e.g., U.S. Pat. Nos. 5,516,964; 5,225,538; 5,428,130; 5,514,582; 5,714,147; and 5,455,165.

### Binding Domain Fusion Proteins

Yet another example of a test agent that can be evaluated practicing the methods and assays of the invention is a binding domain-fusion protein. The term “binding domain fusion protein” as used herein includes a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. Ser. No. 05/0136,049 by Ledbetter, J. et al. for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen.

### Small Molecules

The test agents of the present invention can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al. (1994) J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner, U.S. Pat. No. 5,223,409), spores (Ladner U.S. Pat. No. 5,223,409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

### Recombinant Protein Expression

In certain embodiments, the binding members of the multimeric complexes disclosed herein are proteins found in protein preparations that are produced recombinantly. In addition, test agents evaluated practicing the methods of the invention can be proteins or peptides, e.g., antibody molecules and fusion proteins. The terms “recombinantly expressed protein” and “recombinant protein” as used herein refer to a polypeptide expressed from a host cell that has been manipulated by the hand of man to express that polypeptide. In certain embodiments, the host cell is a mammalian cell. In certain embodiments, this manipulation may comprise one or more genetic modifications. For example, the host cells may be genetically modified by the introduction of one or more heterologous genes encoding the polypeptide to be expressed. The heterologous recombinantly expressed polypeptide can be identical or similar to polypeptides that are normally expressed in the host cell. The heterologous recombinantly expressed polypeptide can also be foreign to the host cell, e.g., heterologous to polypeptides normally expressed in the host cell. In certain embodiments, the heterologous recombinantly expressed polypeptide is chimeric. For example, portions of a polypeptide may contain amino acid sequences that are identical or similar to polypeptides normally expressed in the host cell, while other portions contain amino acid sequences that are foreign to the host cell. Additionally or alternatively, a polypeptide may contain amino acid sequences from two or more different polypeptides that are both normally expressed in the host cell. Furthermore, a polypeptide may contain amino acid sequences from two or more polypeptides that are both foreign to the host cell. In some embodiments, the host cell is genetically modified by the activation or upregulation of one or more endogenous genes.

In another aspect, the invention includes vectors, preferably expression vectors, containing a nucleic acid encoding polypeptides described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein (e.g., binding member proteins, mutant forms thereof, fusion proteins, and the like).

The term “recombinant host cell” (or simply “host cell”), as used herein, is intended to refer to a cell into which a recombinant expression vector has been introduced. It should be understood that such terms are intended to refer not only to the particular subject cell, but to the progeny of such a cell. Because certain modifications may occur in succeeding generations due to either mutation or environmental influences, such progeny may not, in fact, be identical to the parent cell, but are still included within the scope of the term “host cell” as used herein.

The recombinant expression vectors of the invention can be designed for expression of proteins in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be used in activity assays, (e.g., direct assays or competitive assays described in detail below), or to generate antibodies specific for (i.e., against) proteins. In a preferred embodiment, a fusion protein expressed in a retroviral expression vector of the present invention can be used to infect bone marrow cells which are subsequently transplanted into irradiated recipients. The pathology of the subject recipient is then examined after sufficient time has passed (e.g., six weeks).

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The expression vector can be a yeast expression vector, a vector for expression in insect cells, e.g., a baculovirus expression vector or a vector suitable for expression in mammalian cells.

When used in mammalian cells, the expression vector's control functions can be provided by viral regulatory elements. For example, commonly used promoters are derived from polyoma, Adenovirus 2, cytomegalovirus and Simian Virus 40.

In another embodiment, the promoter is an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard (1992) Proc. Natl. Acad. Sci. USA 89:5547, and Paillard (1989) Human Gene Therapy 9:983).

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the □-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus.

Another aspect the invention provides a host cell which includes a nucleic acid molecule described herein, e.g., a nucleic acid molecule within a recombinant expression vector or a nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a protein can be expressed in bacterial cells (such as E. coli), insect cells, yeast or mammalian cells (such as Chinese hamster ovary cells (CHO) or COS cells, CV-1 origin SV40 cells; Gluzman (1981) Cell 23:175-182). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a protein. Accordingly, the invention further provides methods for producing a protein using the host cells of the invention. In some embodiments, the methods include producing (i.e., expressing) full-length protein using the host cells of the invention. In some embodiments, the methods include producing (i.e., expressing) only a soluble receptor domain. In some embodiments, the methods include producing (i.e., expressing) a receptor ectodomain and/or a receptor transmembrane domain. In some embodiments, the methods include producing (i.e., expressing) a binding member antigenic fragment, e.g., a binding member fragment that is capable of interaction with an antibody.

In some embodiments, the method includes culturing the host cell of the invention (into which a recombinant expression vector encoding a protein has been introduced) in a suitable medium such that a protein is produced. In another embodiment, the method further includes isolating a protein from the medium or the host cell.

A number of embodiments of the invention have been described. Nevertheless, it will be understood that various modifications may be made without departing from the spirit and scope of the invention.

### EXAMPLES

The invention is further described in the following examples, which are illustrative and not intended to be limiting the scope of the invention encompassed the claims.

### Example 1

### Analysis of Low Affinity Receptor Binding Affinities Using Surface Plasmon Resonance (SPR)

The precise binding affinities and kinetic parameters of IL-13 and IL-13R110Q to the low affinity receptor, IL-13RI1, were analyzed using a binary heterogeneous assay employing surface Plasmon resonance (SPR).

Human recombinant IL-13, IL-13 was engineered with a C-terminal cysteine residue, as previously described (Yang et al., Anal. Biochem., 351:158-160, 2006), the contents of which are herein incorporated by reference. IL-13R110Q, and a IL-13Rα1 monomer (i.e., the IL-13RI1 extracellular domain) were expressed and purified as described previously (Yang et al., Anal. Biochem., 351:158-160, 2006).

IL-13Rα1 was immobilized on the surface of a research grade CM5 sensor chip, as follows. Each chip was activated using a 30 second pulse of N-ethyl-N-(2-dimethylaminopropyl)carbodiimide hydrochloride mixed with N-hydroxylsuccinimide (NHS-EDC). IL-13RI1 (10 μg/ml) in 10 mM sodium acetate pH 4.0 was then injected over the activated surfaces for one to two minutes to achieve surface densities between 200 and 1160 RU. All surfaces were subsequently deactivated by a 5-minute injection of 1 M ethanolamine-HCl prior to performing kinetic experiments.

Various concentrations of IL-13 or IL-13R110Q ranging from 0.325 nM to 40 nM, or buffer, were prepared in 8.1 mM Na2HPO4, 1.47 mM KH2PO4, pH 7.2, 137 mM NaCl, 2.7 mM KCl, 0.01% BSA, 3.4 mM EDTA and 0.005% Tween 20 (PBS-BET). Each solution of IL-13 and IL-13R110Q was then injected over the surface of the IL-13RI1 coated chip. Measurements were performed at 22° C., 30 μl per minute, and a collection rate of 10 Hz.

All surfaces were regenerated by a 30 second pulse at 60 μl per minute of a solution of 0.549 M MgCl2, 0.138 M KSCN, 0.276 M urea and 0.549 M guanidine-HCl followed by two consecutive 15 second PBS-BET injections. All injections were randomized and performed in triplicate.

Experimental data were corrected for instrumental and bulk artifacts by double referencing a control sensor chip surface and buffer injections using Scrubber software (BioLogic Software v1.1 g) (19). The transformed data were globally fit to 1:1 binding models for experiments with the IL-13Rα1 sensor chip surface or the heterologous ligand binding model for experiments with the IL-13Rα1/IL-4R sensor chip surface using BiaEvaluation v4.1.

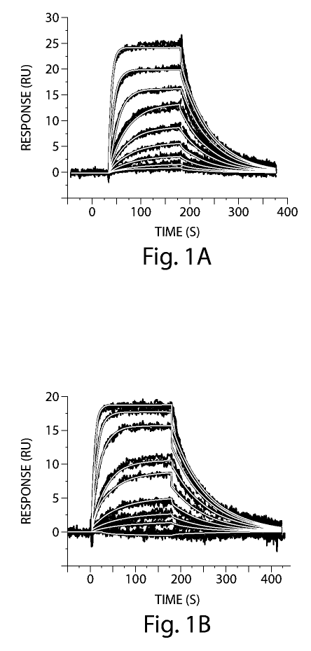

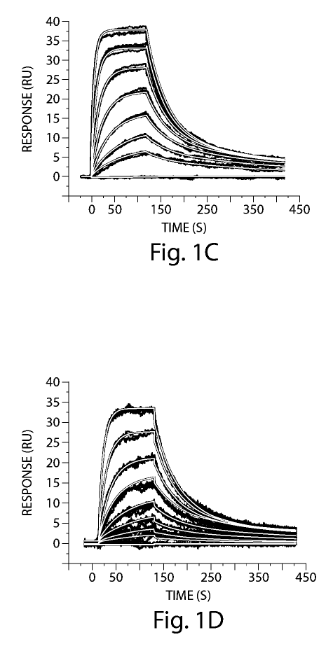

As shown in FIG. 1A (IL-13) and FIG. 1B (IL-13R110Q), the binding profiles of IL-13 and IL-13R110Q were dose-dependent, reached saturation, and at the higher concentrations, reached equilibrium. Briefly, the binding affinity of IL-13 and IL-13R110Q to IL-13Rα1 and IL-13RI1/IL-4R was measured in a label-free system, using a Biacore 3000 instrument. IL-13Rα1 (about 800 RU) was immobilized by direct amine linkage. Various concentrations of (FIG. 1A) IL-13 or (FIG. 1B) IL-13R110Q ranging from 0.325 nM to 40 nM were injected over the Il-13RI1 sensor chip surface. In order to measure the interaction of IL-13 with its high affinity receptor, both IL-13Rα1 (˜600 RU) and IL-4R (˜900 RU) were directly immobilized in a CM5 surface. Various concentrations of IL-13 (FIG. 1C) or IL-13R110Q (FIG. 1D) were injected over the surface. Data shown are triplicate injections of analyte. Data sets for IL-13 and IL-13R100Q fit well to a 1:1 interaction model and were characterized by similar rapid on and off rates.

Table 1 shows the kinetic rates of IL-13 and IL-13R110Q binding to IL-13Rα1 and IL-13Rα1/IL-4R complex. Kinetic rates were determined using the 1:1 model for the IL-13Rα1 sensor chip surface and the heterogeneous ligand model for the IL-13Rα1/IL-4R sensor chip surface in Biaevaluation software v4.1. Data shown are mean and standard deviation from at least 3 independent experiments. As shown in Table 1, the decay of binding, measured from the t½ values, was about 50 seconds. Kd values, calculated from the kinetic parameters of the IL-13 and IL-13R110Q, were 4.9+/−1.3 nM and 8.9+/−1.5 nM, respectively.

This Example demonstrates that both IL-13 and IL-13R110Q bind to IL-13Rα1 with similar affinities and that SPR is a powerful technique to analyze the kinetic properties of such a binary heterogeneous technique.

### Example 2

### Analysis of High Affinity Receptor Binding Affinities Using SPR

The precise binding affinities and kinetic parameters of IL-13 and IL-13R110Q to the high affinity receptor, consisting of IL-13Rα1 and IL-4R, were analyzed using a ternary heterogeneous assay employing SPR. Briefly, to measure IL-13 and IL13R110Q binding to the heterodimeric IL-13 receptor complex (e.g., IL13Rα1 plus IL-4R), a heterogeneous surface was generated that comprised both IL13Rα1 and IL-4R.

Human recombinant IL-13, IL-13R110Q, and IL-13Rα1 monomers are described in Example 1. A soluble form of carrier-free human IL-4Rα (herein referred to as “IL-4R”) monomer was obtained from R&D Systems (Minneapolis, Minn.).

A combination of IL-13Rα1 and IL-4R were co-immobilized on a research grade CM5 sensor chip that was activated as described in Example 1. IL-13Rα1 and IL-4R were then injected separately over the same flow cell for 1-2 minutes, or until surface densities between 200 to 2200 RU were obtained for each receptor. As described above, all surfaces were deactivated by a 5-minute injection of 1 M ethanolamine-HCl prior to performing kinetic experiments.

The ratio of IL-13Rα1 to IL-4R on the heterogeneous surface ranged from 1:1 to 1:10. Association and dissociation rates to each all IL-13Rα1 and IL-4R heterogeneous surfaces were analyzed for a broad range of concentrations of both IL-13 and IL-13R110Q. Injections, measurements, and surface regenerations were performed as described in Example 1.

FIG. 1C shows representative data for a heterogeneous surface with 600 RU IL-13RI1 and 900 RU IL-4R (i.e., a ratio of 1:1.5) exposed to IL-13. FIG. 1D shows representative data for the same heterogeneous surface exposed to IL-13R110Q. As shown in FIGS. 1C and 1D, binding of each IL-13 and IL-13R110Q to heterogenous IL-13Rα1 IL-4R, high affinity receptor, surface reached saturation and was dose-dependent. Furthermore, rate constants observed were not dependent on the ratio of IL-13Rα1 to IL-4R on the surface. Data from several surfaces was, therefore, combined to determine the rate constants shown in Table 1.

As shown in Table 1, for IL-13, the calculated on and off rates and K_{d} for the lower affinity interaction were comparable to those measured directly for IL-13 binding to IL-13Rα1 alone, the low affinity receptor (see Example 1), reflecting both rapid on and off rates. Similarly, the on and off rates and K_{d} calculated for IL-13R110Q were comparable to those measured directly for IL-13R110Q binding to IL-13Rα1 alone (Example 1).

As shown in Table 1, IL-13 binding to the high affinity receptor was characterized by a similar on rate and slower off rate than seen for binding to IL-13Rα1 alone. The slower off rate increased the t½ of the IL-13 molecules dissociation from the IL-4R/IL-13Rα1 surface to about 230 seconds. The resulting calculated Kd was 22-fold lower than to IL-13RI1 alone (0.23 nM). A comparable effect was seen with the IL-13R110Q.

It was determined that the calculated K_{d} for the interaction with IL-4R/IL-13RI1 was reduced 17-fold relative to the IL-13Rα1 alone (0.53 nM). The rates and calculated affinities of both IL-13 and IL-13R110Q were essentially identical (Table 1).

This Example demonstrates the versatility of an heterogeneous assay system to analyze the kinetic properties of complex formation between a receptor and a ligand. This Example also demonstrates that IL-4R does not affect the interaction of IL-13 and/or IL-13R110Q with IL-13Rα1. Thus, this Example is consistent with the functional IL-13 signaling complex formation reported in the art.

### Example 3

### Kinetics of IL-13 Signaling Receptor Formation Using SPR

As described above, the binding of IL-4R to the binary IL-13/IL-13Rα1 complex to form a ternary IL-13/IL-13Rα1/IL-4R ternary complex is required for IL-13 mediated biological signaling. To characterize the formation of this ternary complex, we directly measured the association and dissociation of IL-4R binding to a preformed IL-13 or IL-13R110Q/IL-13Rα1 binary complex using a ternary heterogeneous assay employing SPR.

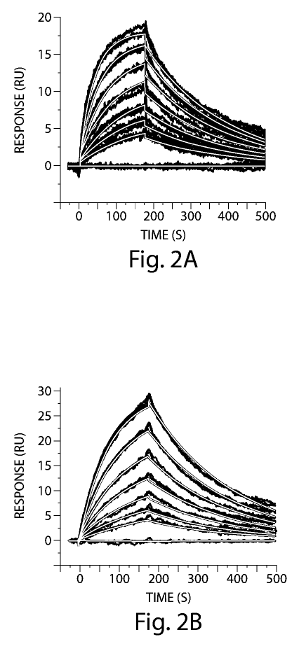

Briefly, IL-13Rα1 was immobilized on the sensor chip surface as described in Example 1. A constant amount (8 nM) of IL-13 or IL-13R110Q was added to the running buffer and sample buffers to form the binary complex (as shown in FIGS. 2A and 2B, respectively). Various concentrations of IL-4R were then injected to the binary complex. Injections, measurements, and surface regenerations were performed as described in Example 1.

FIGS. 2A-2B are line graphs showing SPR measurements of IL-4R binding kinetics to the IL-13/IL-13Rα1 binary complex. Response units monitored in real time for various dilutions of IL-4R (0 to 400 nM) after injection on either (A) IL-13/IL-13Rα1 or (b) IL-13R110Q/IL-13Rα1 binary complex coated on the surface of a heterogeneous sensor chip surface. For each graph the data, shown in the black wavy lines, are triplicate measurements for each concentration. The calculated fit from a 1:1 model using BiaEval software v4.1 is shown using a solid red line. Each data set is representative of 3 independent experiments.

IL-13 does not significantly bind to IL-4R directly, but first binds IL-13Rα1 and this complex binds IL-4R. In order to measure the kinetic parameters of this interaction, IL-13Rα1 was first directly immobilized on a CM5 chip (˜500 RU), then 8 nM IL-13 or IL-13R110Q was included in the running buffer and sample buffers to establish a stable complex of IL-13/IL-13Rα1. Various dilutions of IL-4R starting at 400 nM were injected on the (FIG. 2A) IL-13/IL-13Rα1 or (FIG. 2B) IL-13R110Q/IL-13Rα1 complexes.

TABLE 3
Binding Kinetics of IL-4R Binding to the IL-13/IL-13Rα1 Complex

| | Analyte - 1 | | | | |
|---|---|---|---|---|---|
| Ligand | (constant, | Analyte - 2 | Kon × | Koff | KD |
| (immobilized) | in buffer) | (Injections) | 106 M−1 s−1 | 1/s | nM |
| IL-13Rα1 | IL-13 | IL-4R | .063 ± .006 | .0045 ± .0001 | 71.6 ± 5.5 |
| | (8 nM) | | | | |
| IL-13Rα1 | IL- | IL-4R | .044 ± .001 | .0051 ± .0001 | 115 ± 5.5 |
| | 13R110Q | | | | |
| | (8 nM) | | | | |

IL-4R binding to the binary complex was dose-dependent and fit well to a 1:1 model. As shown in Table 1, IL-4R binding was observed to have a relatively slow association rate of 0.063+/−0.006×10⁶ M⁻¹s⁻¹ and a relatively slow dissociation rate of 0.0045+/−0.0001 s⁻¹ with a calculated K_{d} of 71.6+/−5.5 nM. The decay of binding, measured from the t½ value was about 150 seconds. The binding kinetics of IL-4R to IL-13R110Q/IL-13Rα1 (kₒₙ=0.044+/−0.001×10⁶ M⁻¹s⁻¹, k_{off}=0.0051+/−0.0001 s⁻¹, K_{d}=115+/−5.5 nM) was essentially the same, suggesting that the association of the variant IL-13R110Q with human asthma and elevated IgE levels is not likely due to differences in binding affinity in the IL-13R-IL-4R complex.

This Example demonstrates that IL-13 ternary complex formation occurs on a heterogeneous surface using the methods described herein. This Example also demonstrates that IL-4R binds to the IL-13 or IL-13R110Q/IL-13Rα1 binary complex to form a IL-13 or IL-13R110Q/IL-13Rα1/IL-4R ternary complex and that a heterogeneous assay system can be used to analyze the kinetic properties of the formation of a this ternary complex.

The dissociation constants observed using the SPR techniques described herein are similar to those values reported by measuring IL-13 binding to cells that express the high affinity signaling receptor complex (Aman et al., J. Biol. Chem., 271:29265-70, 1996; Hilton et al., Proc. Natl. Acad. Sci., 93:497-501, 1996; Caput et al., J. Biol. Chem., 271:16921-6, 1996; Miloux et al., FEBS Lett., 401:163-6, 1997). The similar values of the dissociation constant of IL-13 between SPR measurements, using soluble, monomeric forms of the receptor components, and cells expressing the full length proteins suggests that all binding among these components occurs in the extracellular portion of the receptors.

In addition, although IL-4R increases the binding affinity of IL-13 in the ternary complex (i.e. IL-13/IL-13Rα1/IL-4R) compared to the binary complex, IL-4R binds the binary complex with a relatively slow on rate and a fast off rate, resulting in a weak dissociation constant of ˜100 nM. The slow on rate of IL-4R binding supports the hypothesis that IL-13 binding to IL-13Rα1 induces a conformational change that allows binding to IL-4R (Moy et al., J. Mol. Biol., 310:219-230, 2001).

### Example 4

### Time-Resolved Fluorescence Resonance Energy Transfer Assay

Two versions of a homogeneous TR-FRET assay (designated TR-FRET assay 1 and TR-FRET assay 2) were developed to analyze the interactions between IL-13 and/or IL-13R110Q, IL-13Rα1, and IL-4R without the need for the immobilization of a molecule or combination of molecules on a heterogeneous surface.

A—TR-FRET Assay 1

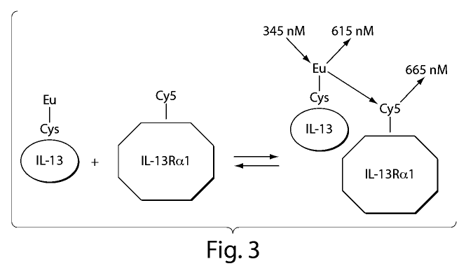

TR-FRET assay 1 is a bimolecular assay that involves europium chelate (Eu) labeled IL-13 (Eu-IL-13) and Cy5 labeled IL-13RI1 (Cy5-IL-13Rα1). In this system, the Eu label is the donor probe and Cy5 is the acceptor molecule. As shown in FIG. 3, TR-FRET assay 1 can be used as a bimolecular assay (i.e., IL-13 and IL-13Rα1) alone or in the presence of unlabeled IL-4R.

B—TR-FRET Assay 2

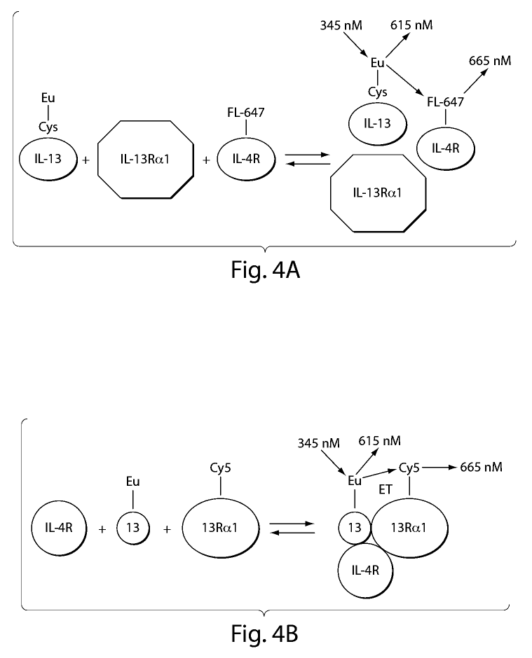

TR-FRET assay 2 is a ternary assay that involves Eu-IL-13, Alexa Fluor 647 (FL647) labeled IL-4R (IL-4R-FL647), and unlabeled IL-13Rα1. In this system, the Eu label is the donor probe and FL647 is the acceptor molecule. As shown in FIG. 4, in the ternary assay, in the absence of FL647, Eu is detected at 615 nm and the TR-FRET signal, which is emitted at 665 nm, is totally dependent on the formation of the IL-13 (or IL-13R110Q)/IL-13Rα1/IL-4R ternary complex. The IL-13 ternary complex, and thus the TR-FRET signal, will not be formed in the absence of unlabeled IL-13Rα1.

C—Direct Protein Labeling

IL-13 and IL-13Rα1 were directly labeled as previously described with some modifications (Yang et al. (2006) Analytical Biochemistry, 351:158-160).

IL-13 was labeled with the donor molecule, Europium chelate (Eu) (Perkin Elmer, Wilton, Conn.) and IL-13Rα1 was labeled with the acceptor molecule Cy5 (Perkin Elmer). IL-4R was labeled with the Cy5 equivalent FL647 (Invitrogen, Carlsbad, Calif.), which serves as a TR-FRET acceptor. FL647 labeling was performed using a kit according to the manufacturer's instructions with slight modifications to better suit the small amounts of protein labeled in this example. Briefly, the IL-4R was reconstituted in 100 μM bicarbonate buffer (pH 8.3), to a final protein concentration of 1.0 mg/ml, mixed with the FL647 dye, and incubated at room temperature for one hour. Unincorporated FL647 dye was separated from the IL-4R using a micro column.

Hereafter, the labeled proteins were referred to as Eu-IL-13, Cy5-IL-13Rα1 and IL-4R-FL647.

All TR-FRET experiments were performed on a 384-well black plate (Corning Costar, Acton, Mass.) in 20 μL final volume of PBS plus 0.1% BSA. Excitation and emission conditions were the same for TR-FRET assay 1 and 2, as indicated in FIGS. 3 and 4, respectively. For example, excitation and emission conditions for TR-FRET assay 1 and 2 were 345 nm and 665 nm, respectively. All TR-FRET measurements were taken using an Envision plate reader (Perkin Elmer) using the TR-FRET mode.

This Example demonstrates the techniques required to directly label components of the IL-13 ternary complex with molecules suitable for excitation and detection using TR-FRET.

### Example 5

### Analysis of IL-13 Binding Affinity to IL-13RI1 Using Bimolecular TR-FRET Assay 1

Binding between IL-13 and IL-13Rα¹ was analyzed using Eu-IL-13 (Eu=Europium chelate, FRET donor) and Cy5-IL-13Rα1 (Cy5=Cyanine dye, FRET acceptor) (schematic shown in FIG. 3) described in Example 5. Affinity measurements were performed by adding various concentrations of Cy5-IL-13Rα1 in the presence of 10 nM Eu-IL-13 with and without 500 nM IL-4R.

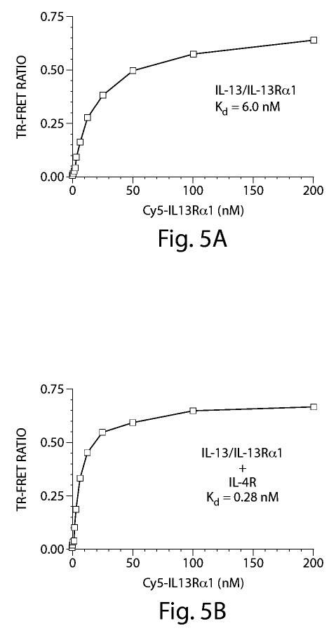

As shown in FIG. 5A, the binding between Eu-IL-13 and Cy5-IL-13Rα1 reached saturation, with the half-maximal TR-FRET signal occurring at 10 nM. As shown in Table 1, the calculated dissociation constant was 6 nM (Table 1).

As shown in FIG. 5B, ternary complex formation was observed following the addition of 500 nM IL-4R to the homogeneous reaction. For these experiments, binding reached saturation as indicated in FIG. 5B. The binding constant was estimated to be 0.28 nM using Equation (1) (Table 1). Because the measured K_{d} is much smaller than the concentration of Eu-IL-13 used in the reaction, the binding affinity is subject to large error. Nevertheless, the difference between IL13-Rα1 binding in the presence (FIG. 5B) or absence (FIG. 5A) of IL-4R is apparent.

This Example demonstrates that IL-4R increases the binding affinity of IL-13 in the ternary complex compared to the binary complex.

### Example 6

### Analysis of IL-13 and IL-13R110Q Binding Affinities to IL-13RI1 Using Bimolecular TR-FRET Assay 1

The affinities of IL-13 and IL-13R110Q binding to IL-13Rα1 were compared using a competition assay for the binary complex.

Assays were performed as described in Example 5. Competition experiments were performed by adding various concentrations of unlabeled IL-13, IL-13R110Q, or IL-13Rα1 to the Eu-IL-13 (10 nM)/Cy5-IL-13Rα1 (10 nM) binary complex with or without IL-4R (500 nM).

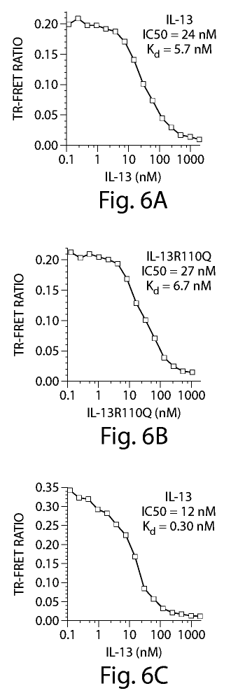

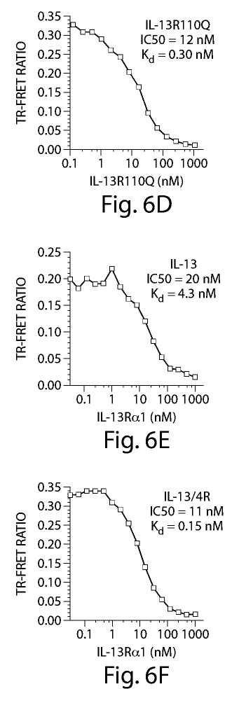

As shown in FIGS. 6A and 6B, increasing concentrations of IL-13 (6A) or IL-13R110Q (6B) competed the binding of the Eu-labeled IL-13 to Cy5-IL-13Rα1. The decrease in TR-FRET signal was dose-dependent and reached background levels at the highest concentrations for each IL-13 and the R110Q variant. IC50 values were 24 and 27 nM, respectively.

The dissociation constant, calculated using Equation (1) was 5.7 nM for IL-13, essentially identical to that determined by direct binding of Eu-labeled IL-13 to IL-13Rα1 (6.0 nM) (Table 1).

This result confirms the equivalence of the unlabeled and labeled IL-13. The dissociation constant for IL-13R110Q was 6.7 nM, indistinguishable from that for IL-13 (Table 1). Thus, this Example demonstrates that, using the novel homogeneous format, IL-13 and IL-13R110Q bind with equivalent affinity to IL-13RI1.

### Example 7

### Determination of Dissociation Constants for IL-13 and IL-13R110Q Using Bimolecular TR-FRET Assay 1

Dissociation constants of IL-13 and IL-13R110Q in the formation of the tertiary complex were analyzed using the competition experiments described in Example 6.

As shown in FIGS. 6C and 6D, increasing concentrations of IL-13 (C) or IL-13R110Q (D) that were added to fixed concentrations of Eu-IL-13, Cy5-IL-13Rα1, and IL-4R showed a dose-dependent inhibition of the TR-FRET signal with complete inhibition at the highest concentrations of cytokine. Dose response curve yielded IC50 values of 12 nM for both proteins.

The binding isotherm was consistent with competition by a single species. A K_{d} of 0.30 nM was calculated from the IC50 value for both IL-13 and IL-13R110Q (Table 1). These results confirm that IL-13 and IL-13R110Q have indistinguishable binding properties in the formation of the ternary complex.

To confirm the dissociation constant of Cy5-IL-13Rα1, a competition experiment was performed using unlabeled IL-13Rα1. In the presence of 10 nM each Eu-IL-13 and Cy5-IL-13Rα1, with and without 500 nM IL-4R, various concentrations of unlabeled IL-13Rα1 ranging from 0 to 1000 nM showed dose-dependent inhibition and reached complete inhibition at the highest concentrations.

As shown in FIG. 6E, in the absence of IL-4R, an IC50 of 20 nM was observed, corresponding to a K_{d} of 4.3 nM, which compares well with the 6.0 nM K_{d} from the direct binding measurement (Table 1). This observation indicates that the Cy5-IL-13Rα1 has indistinguishable binding compared to the unlabeled receptor.

As shown in FIG. 6F, in the presence of 500 nM IL-4R, an IC50 of 11 nM was observed, corresponding to a K_{d} of 0.15 nM, which compares to the 0.28 nM measured from the direct binding assay format shown in FIG. 5B and Table 1.

This Example demonstrates that the kinetic properties observed using SPR and TR-FRET are highly consistent. As described above, the kinetic properties observed using SPR were also highly consistent to values previously reported using cell surface studies. Thus, the immobilization and labeling of the various components of the IL-13 receptor signaling complex does not evoke artificial conformational changes in any of the components of the IL-13 ternary complex.

### Example 8

### Determination of Dissociation Constant for IL-4R Binding to the IL-13/IL-13Rα1 Binary Complex Using Ternary TR-FRET Assay 2

The binding affinity between IL-4R and the binary complex of IL-13 and IL-13Rα1 was measured between IL-4R-FL647 and Eu-IL-13 in the presence of unlabeled IL-13Rα1. The TR-FRET signal was monitored in samples containing a final concentration of 20 nM each, Eu-IL-13, and IL-13Rα1, and various concentrations of IL-4R-FL647 ranging from 0 to 1100 nM. Based on the observations described above, 60% of the Eu-IL-13 and IL-13Rα1 was predicted to associate in the absence of IL-4R. Likewise, in the presence of IL-4R, the binding percentage was predicted to increase, since bringing IL-4R to the complex increases the binding affinity of IL-13 and IL-13Rα1.

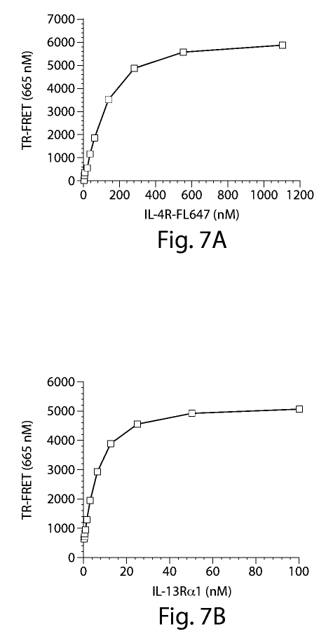

As shown in FIG. 7A, various concentrations of IL-4R-FL647 added to IL-13Rα1 showed the predicted dose-dependent TR-FRET signal and reached saturation at the higher IL-4R-FL647 concentrations. Curve fitting of the dose response data yielded a K_{d} value of 100 nM for IL-4R (Table 1).

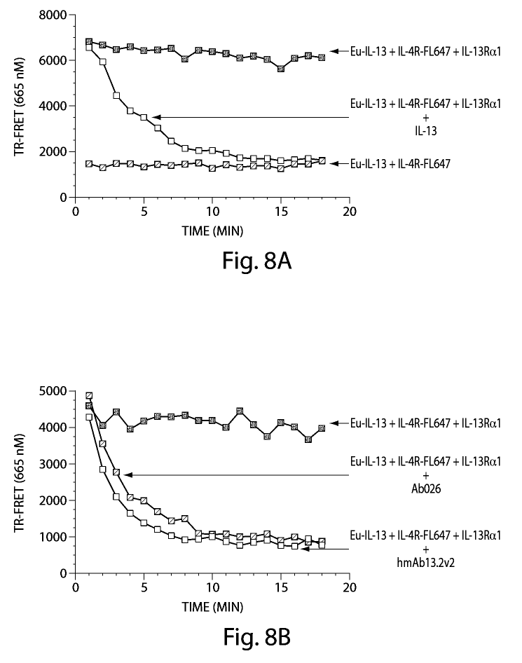

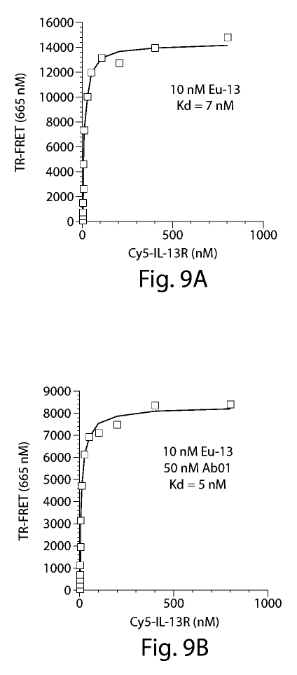

As shown in FIG. 8A, samples without IL-13RI1 did not show any TR-FRET signal due to direct binding between Eu-IL-13 and IL-4R-FL647 (labeled Eu-IL-13+IL-4R-FL647). These results are consistent with SPR binding studies described above. However, as shown in FIG. 8A, in the absence of IL-13Rα1, significant background signal was observed at the highest concentrations of IL-4R-FL647 due to optical energy transfer from the Eu-IL-13 emission at 615 nm to IL-4R-FL647. A competition study using unlabeled IL-4R was not conducted due to the large amount of reagent required. However, it was confirmed that this background signal was not due to binding between Eu-IL-13 and IL-4R-FL647, since it could not be inhibited by unlabeled IL-13. Thus the true TR-FRET signal, due to the binding of IL-4R-FL647 to the Eu-IL-13/IL-13Rα1, complex was determined as the difference in fluorescence at 665 nm of samples in the presence and absence of IL-13Rα1.

### Example 9

### Optimization and Validation of the Ternary TR-FRET Assay 2

The dose-dependent IL-4R TR-FRET signal generated by association of the ternary complex validated a potential assay for monitoring inhibition of IL-4R binding to the IL-13/IL-13RI1 complex. This assay may be useful to identify molecules that inhibit IL-13 function by blocking either IL-13 binding to IL-13Rα1, or that inhibit the binary complex binding to IL-4R. To establish optimal conditions for an IL-4R binding assay, experiments were performed to establish an IL-13Rα1 concentration that yielded a broad dynamic range while maintaining Eu-IL-13 as the limiting reagent.

In order to optimize the TR-FRET signal under fixed concentrations of labeled reagents (Eu-IL-13 and IL-4R-FL647), various concentrations of IL-13Rα1 (unlabeled) was added to find the least amount of IL-13Rα1 that yields a broad dynamic assay window and also keeps Eu-IL-13 as the limiting reagent in order to minimize background signal. Since the TR-FRET complex consists of three proteins, the TR-FRET signal intensity depends not only the binding between Eu-IL-13 and IL-13Rα1 (the binary complex), but also the binding between IL-4R-FL647 and the binary complex. As mentioned above, with 20 nM of both Eu-IL-13 and IL-13Rα1, 60% of the FRET donor is bound to form the binary complex as determined by analyzing the K_{d}. The final concentration of the TR-FRET complex also depends on the concentration of IL-4R-FL647. In experiments performed to determine the EC90 value, the final concentration in the assay was 20 nM Eu-IL-13, 200 nM IL-4R-FL647, and increasing concentrations of IL-13Rα1 ranging from 0 to 200 nM.

As shown in FIG. 7B, an EC90 was reached at 25 nM IL-13Rα1. Under these conditions, about 50% of Eu-IL-13 was bound to form the TR-FRET complex and only about 5% of the IL-4R-FL647 was bound in the complex. Thus, 25 nM of IL13Rα1 provides a sufficient TR-FRET signal intensity to monitor inhibition of IL-4R binding.

To confirm that the observed TR-FRET signal was generated from a specific interaction between the two labeled proteins in the presence of unlabeled IL-13Rα1, a competitive binding experiment was performed using unlabeled IL-13 with a pre-formed ternary complex (i.e., IL-13/IL-13Rα1/IL-4R). Eu-IL-13 and Il-13Rα1 was mixed with IL-4R-FL647 to form the TR-FRET complex. Eu-IL-13 and IL-4R-FL647 were mixed in the absence of IL-13RI1 as a negative control. Unlabeled IL-13 was then added to the homogeneous assay to determine if unlabeled IL-13 was capable of reducing the TR-FRET signal by disrupting the ternary complex. Unlabeled IL-13 was added to a final concentration of 3.0 μM and the TR-FRET signal was measured in a kinetic mode using the Envision plate reader.

As shown in FIG. 8A, unlabeled IL-13 decreased the TR-FRET signal in a time-dependent manner and the signal reached background in about 12 minutes. Furthermore, the TR-FRET signal was low in samples without IL-13Rα1 and no change occurred with addition of IL-13, confirming the absence of binding between Eu-IL-13 and IL-4R-FL647. Samples with IL-13Rα and without the added unlabeled IL-13 maintained a strong TR-FRET signal.

Similar experiments where then performed using the humanized antibody, hmAb13.2v2, and antibody Ab026 as the competing agents in place of unlabeled IL-13. mAb 13.2 and its humanized form hmAb13.2v2 are described in commonly owned U.S. application U.S. Ser. No. 06/0063,228 or its PCT application WO 05/123126, the contents of which are incorporated herein by reference in their entirety. Ab026 (also referred to as “MJ2-7”) and humanized versions thereof are described in commonly owned US application 2006/0073148, the contents of which are also incorporated herein by reference in their entirety.

Each of these two antibodies binds to different components of the ternary complex. On the one hand, antibody hmAb13.2v2, binds to IL-13 and blocks IL-4R binding to the IL-13/IL-13RI1 binary complex. Note hmAb13.2v2 does not prevent or disrupt the formation of the binary complex. On the other hand, antibody Ab026, binds to IL-13 and prevent IL-13 from binding IL-13Rα1. Thus, Ab026 is believed to prevent the formation of the binary complex. Despite these different mechanisms of action, both antibodies block the functional response of IL-13 by disrupting or preventing the formation of the ternary complex.

200 nM of both hmAb13.2v2 and Ab026 were added to a homogeneous assay containing a preformed IL-13 ternary complex to determine if either antibody was capable of reducing the TR-FRET signal by disrupting the ternary complex.

As shown in FIG. 8B, the addition of either hmAb13.2v2 or Ab026 considerably reduced the pre-formed TR-FRET signal in a time-dependent manner. Therefore, these results indicate that blocking either IL-4R-FL-647 binding to the binary complex or Eu-IL-13 binding to IL-13Rα1 can be detected using this novel homogeneous assay.

Thus, TR-FRET assay data is in agreement with the data obtained using a heterogeneous SPR assay format. Thus, the TR-FRET assay described herein provides a homogeneous assay format for characterizing interactions of IL-13 and its receptor components. The assay is rapid, robust, and uses minimal amounts of proteins. As described above, this assay has been shown to be useful for characterizing and comparing IL-13 and IL-13R110Q binding to IL-13Rα1 and has demonstrated that the there is no difference in the binding to IL-13Rα1 or the binary complex for IL-13 and IL-13R110Q. In other words, IL-13R110Q has the same binding affinity in both the binary and ternary complex as IL-13. These findings indicate that the above described association of IL-13R110Q with human asthma and elevated IgE levels is not likely due to differences in binding affinity in the IL-13Rα1/IL-4R complex. Similar approaches to those described herein could be used to characterize other IL-13 or receptor variants. For example, binding affinities for the complex could be examined using variants of IL-13Rα1 and/or IL-4R.

The data presented herein also demonstrate that the TR-FRET assays described herein can be used to screen for molecules that inhibit IL-13 either by blocking IL-13 binding to IL-13Rα1 or by blocking IL-4R binding to the binary complex. Many cytokine receptors are made up of multiple chains. Thus, the TR-FRET assays described herein can be adapted to characterize multimeric interactions for other cytokine receptor complexes. These methods are readily adaptable to high throughput screening and can be engineered for use with a wide variety of assays, for example using microplate readers.

TABLE 1
Rate Constants and Calculated Dissociation Constants Determined By
Surface Plasmon Resonance and Time-Resolved Fluorescence Resonance Energy
Transfer

| | | | | Kon | | | | |
|---|---|---|---|---|---|---|---|---|
| | | | K_{d} 1 | 1 × 10⁶ | K_{off} 1 | K_{d} 2 | K_{d} 2 × 10⁶ | K_{off} 2 |
| | Receptor Complex | Analyte | nM | (M⁻¹s⁻¹) | (s⁻¹) | nM | (M⁻¹s⁻¹) | (S⁻¹) |
| SPR | IL-13Rα1 | IL-13 | 4.88 ± 1.3 | 2.87 ± 0.72 | 0.014 ± .001 | — | — | — |
| | IL-13Rα1 | IL13- | 8.93 ± 1.4 | 1.68 ± 0.20 | 0.015 ± .001 | — | — | — |
| | | R110Q | | | | | | |
| | IL-13Rα1 + IL-4R | IL-13 | 7.39 ± 1.1 | 3.79 ± 1.0 | 0.028 ± .004 | 0.23 ± 0.1 | 13.8 ± 3.3 | 0.003 ± .001 |
| | IL-13Rα1 + IL-4R | IL- | 14.5 ± 2.8 | 1.24 ± 0.27 | 0.018 ± .003 | 0.53 ± 0.2 | 3.77 ± .56 | 0.002 ± .001 |
| | | 13R110Q | | | | | | |
| | IL-13Rα1 + IL-13 | IL-4R | 71 ± 5.5 | 0.063 ± .006 | .004 ± .0001 | — | — | — |
| | IL-13Rα1 + IL- | IL-4R | 115 ± 5.5 | 0.044 ± .001 | .005 ± .0001 | — | — | — |
| | 13R110Q | | | | | | | |

FRET

| | | | | | | | | |
|---|---|---|---|---|---|---|---|---|
| D | Cy5-IL-13/Rα1 | Eu-IL-13 | 6 | — | — | — | — | — |
| C | Eu-IL-13/Cy5-IL- | IL-13 | 5.7 | — | — | — | — | — |
| | 13Rα1 | | | | | | | |
| C | Eu-IL-13/Cy5-IL- | IL-13 | 6.7 | — | — | — | — | — |
| | 13Rα1 | R110Q | | | | | | |
| C | Eu-IL-13/IL-4R/Cy5- | IL-13 | — | — | — | 0.3 | — | — |
| | IL-13Rα1 | | | | | | | |
| C | Eu-IL-13/IL-4R/Cy5- | IL-13 | — | — | — | 0.3 | — | — |
| | IL-13Rα1 | R110Q | | | | | | |
| D | IL-4R-FL-647/Eu-IL- | Cy5-IL- | — | — | — | 0.28 | — | — |
| | 13 | 13RI1 | | | | | | |
| C | Eu-IL-13/Cy5- | IL- | 4.3 | — | — | — | — | — |
| | IL13Rα1 | 13RI1 | | | | | | |
| C | Eu-IL-13/Cy5-IL- | IL- | — | — | — | 0.15 | — | — |
| | 13Rα1/IL-4R | 13RI1 | | | | | | |
| D | Eu-IL-13/Cy5-IL- | IL-4R- | 100 | — | — | — | — | — |
| | 13Rα1 | FL-647 | | | | | | |

(D) = Direct;
(C) = Competition;
(—) = Data not acquired;
K_{d} = Dissociation constant.

Table 1 Data Analysis

SPR

For SPR measurements, rate constants were determined using a 1:1 model for the IL-13RI1 sensor chip surface and a heterogeneous ligand model for the IL-13Rα1/IL-4R sensor chip surface in Biaevaluation software v4.1. Data shown are mean and standard deviation from at least 3 independent experiments.

TR-FRET

Homogeneous TR-FRET K_{d} calculations were performed using two different methods. Method 1 was used for direct binding experiments, and method 2 was used for competitive experiments.

Method 1—Direct Binding Experiments

Data were fitted to the bimolecular binding model presented in Equation (1):

$$[ RL ] = \frac{{[ L ]}_{t} + {[ R ]}_{t} + {K}_{d} - {\{ {( {[ L ]}_{t} + {[ R ]}_{t} + {K}_{d} )}^{2} - {{4 [ R ]}_{t} [ L ]}_{t} \}}^{1 / 2}}{2}$$

wherein [RL], [L]ₜ, and [R]ₜ are the concentrations of the complex, total ligand, and total receptor, respectively. K_{d} is the dissociation constant.

Method 2—Competition Experiments

The measurements taken for competition experiments were IC50 values. These values were converted to Kᵢ using the exact relation between Kᵢ and IC50 according to Equation (2):

$${K}_{i} = \frac{F}{2 - F} {K}_{d} \{ \frac{IC 50}{( {[ R ]}_{t} - {K}_{d} \frac{F}{2 - F} - {\frac{F}{2} [ L ]}_{t} )} - 1 \}$$

wherein F is the bound fraction of the labeled ligand in the absence of a competitor. K_{d} and Kᵢ are the dissociation constants of labeled and unlabeled ligand, respectively. Where K_{d} is known, Kᵢ was calculated using the IC50, according to Equation (2).

Where K_{d} is unknown, the relationship between K_{d} and Kᵢ was determined, as follows.

K_{d}=Kᵢ when a labeled reagent is identical to its corresponding unlabeled counterpart in competition experiments. It is assumed that IC50 values for unlabeled reagents are equal to the K_{d} values measured in the direct binding experiments, assuming, of course, that labeled and unlabeled proteins have equal binding affinities. Based on these assumptions, the K_{d} of a component in the formation of a complex was measured using competition experiments.

Note, even where K_{d} is unknown, it is equal to Ki, as stated above. Equation (2), therefore, has only one unknown (K_{d} or Kᵢ), which was solved from a single value of IC50 by plotting Kᵢ vs. a range of K_{d} values, within a range according to Equation (2), using the measured IC50 value. In this scenario, the K_{d} value that gave an equal Kᵢ (where Kᵢ=K_{d}) was the K_{d} for the component of interest.

TABLE 2
EXAMPLES OF FLUOROPHORES

| | Excitation | Emission |
|---|---|---|
| FLUOROPHORE | (nm) | (nm) |
| 1,5 IAEDANS | 336 | 490 |
| 1,8-ANS | 372 | 480 |
| 4-Methylumbelliferone | 385 | 502 |
| 5-carboxy-2,7-dichlorofluorescein | 504 | 529 |
| 5-Carboxyfluorescein (5-FAM) | 492 | 518 |
| 5-Carboxynapthofluorescein | 512/598 | 563/668 |
| 5-Carboxytetramethylrhodamine (5-TAMRA) | 542 | 568 |
| 5-FAM (5-Carboxyfluorescein) | 492 | 518 |
| 5-HAT (Hydroxy Tryptamine) | 370-415 | 520-540 |
| 5-ROX (carboxy-X-rhodamine) | 578 | 604 |
| | 567 | 591 |
| 5-TAMRA (5-Carboxytetramethylrhodamine) | 548 | 552 |
| | 542 | 568 |
| 6-Carboxyrhodamine 6G | 518 | 543 |
| 6-CR 6G | 518 | 543 |
| 6-JOE | 520 | 548 |
| 7-Amino-4-methylcoumarin | 351 | 430 |
| 7-Aminoactinomycin D (7-AAD) | 546 | 647 |
| 7-Hydroxy-4-methylcoumarin | 360 | 449, 455 |
| 9-Amino-6-chloro-2-methoxyacridine | 412 | 471 |
| | 430 | 474 |
| ABQ | 344 | 445 |
| Acid Fuchsin | 540 | 630 |
| ACMA (9-Amino-6-chloro-2-methoxyacridine) | 412 | 471 |
| | 430 | 474 |
| Acridine Orange | 520 | 526 |
| | 460 | 650 |
| Acridine Red | 455-600 | 560-680 |
| Acridine Yellow | 470 | 550 |
| Acriflavin | 436 | 520 |
| Acriflavin Feulgen (SITSA) | 355-425 | 460 |
| Alexa Fluor 350 ™ | 346 | 442 |
| | 342 | 441 |
| Alexa Fluor 430 ™ | 431 | 540 |
| Alexa Fluor 488 ™ | 495 | 519 |
| | 492 | 520 |
| Alexa Fluor 532 ™ | 531 | 553 |
| | 532 | 554 |
| Alexa Fluor 546 ™ | 556 | 572 |
| | 557 | 573 |
| Alexa Fluor 568 ™ | 577 | 603 |
| | 578 | |
| Alexa Fluor 594 ™ | 590 | 617 |
| | 594 | 618 |
| Alexa Fluor 633 ™ | 632 | 650 |
| Alexa Fluor 647 ™ | 647 | 666 |
| Alexa Fluor 660 ™ | 668 | 698 |
| Alexa Fluor 680 ™ | 679 | 702 |
| Alizarin Complexon | 530-560 | 624-645 |
| Alizarin Red | 530-560 | 580 |
| Allophycocyanin (APC) | 630-645 | 655-665 |
| APC-Cy7 | 625-650 | 755 |
| AMC, AMCA-S | 345 | 445 |
| AMCA (Aminomethylcoumarin) | 345 | 425 |
| | 347 | 444 |
| AMCA-X | 353 | 442 |
| Aminoactinomycin D | 555 | 655 |
| Aminocoumarin | 346 | 442 |
| | 350 | 445 |
| Aminomethylcoumarin (AMCA) | 345 | 425 |
| | 347 | 444 |
| Anthrocyl stearate | 360-381 | 446 |
| APTS | 424 | 505 |
| Astrazon Brilliant Red 4G | 500 | 585 |
| Astrazon Orange R | 470 | 540 |
| Astrazon Red 6B | 520 | 595 |
| Astrazon Yellow 7 GLL | 450 | 480 |
| Atabrine | 436 | 490 |
| ATTO-TAG ™ CBQCA | 465 | 560 |
| ATTO-TAG ™ FQ | 486 | 591 |
| Auramine | 460 | 550 |
| Aurophosphine G | 450 | 580 |
| Aurophosphine | 45-490 | 515 |
| BAO 9 (Bisaminophenyloxadiazole) | 365 | 395 |
| BCECF (high pH) 492, 503 520, 528 | 492 | 520 |
| | 503 | 528 |
| BCECF (low pH) | 482 | 520 |
| Berberine Sulphate | 430 | 550 |
| Beta Lactamase | 409 | 447-520 |
| BG-647 | | |
| Blue Fluorescent Protein | 381 | 445 |
| | 382 | 447 |
| | 383 | 448 |
| Bimane | 398 | 490 |
| Bisbenzamide | 360 | 461 |
| Blancophor FFG | 390 | 470 |
| Blancophor SV | 370 | 435 |
| BOBO ™-1 | 462 | 481 |
| BOBO ™-3 | 570 | 602 |
| Bodipy | 492-591 | 509-676 |
| Bodipy Fl | 504 | 511 |
| | 505 | 513 |
| Bodipy FL ATP | 505 | 514 |
| Bodipy Fl-Ceramide | 505 | 511 |
| Bodipy R6G SE | 528 | 547 |
| Bodipy TMR | 542 | 574 |
| Bodipy TMR-X conjugate | 544 | 573 |
| Bodipy TMR-X, SE | 544 | 570 |
| Bodipy TR | 589 | 617 |
| Bodipy TR ATP | 591 | 620 |
| BO-PRO ™-1 | 462 | 481 |
| Brilliant Sulphoflavin FF | 430 | 520 |
| Calcein 494 517 | 494 | 517 |
| Calcein Blue | 373 | 440 |
| Calcium Crimson ™ | 588 | 611 |
| | 589 | 615 |
| Carboxy-X-rhodamine (5-ROX) | 576 | 601 |
| Cascade Blue ™ | 377-399 | 420-423 |
| Catecholamine | 410 | 470 |
| CFDA | 494 | 520 |
| CFP—Cyan Fluorescent Protein | 430-453 | 474-501 |
| Chlorophyll | 480 | 650 |
| Chromomycin A | 436-460 | 470 |
| Chromomycin A | 445 | 575 |
| Coelenterazine O | 460 | 575 |
| Coumarin Phalloidin | 387 | 470 |
| Cy2 ™ | 489 | 506 |
| Cy3.1 8 | 554 | 568 |
| Cy3.5 ™ | 581 | 598 |
| Cy3 ™ | 514 | 566 |
| | 552 | 570 |
| | 554 | |
| Cy5.1 8 | 649 | 666 |
| Cy5.5 ™ | 675 | 695 |
| Cy5 ™ | 649 | 666 |
| Cy7 ™ 710, 743 767, 805 | 710 | 767 |
| | 743 | 805 |
| Cyan GFP | 433 (453) | 475 (501) |
| Cyclic AMP Fluorosensor (FiCRhR) | 500 | 517 |
| Dansyl | 340 | 578 |
| Dansyl Amine | 337 | 517 |
| Dansyl Cadaverine | 335 | 518 |
| DAPI | 359 | 461 |
| Dapoxy 1 | 403 | 580 |
| Dapoxyl 2 | 374 | 574 |
| Dapoxyl 3 | 373 | 574 |
| DCFDA | 504 | 529 |
| DCFH (Dichlorodihydrofluorescein Diacetate) | 505 | 535 |
| DDAO | 463 | 607 |
| DHR (Dihydorhodamine 123) | 505 | 534 |
| Di-4-ANEPPS | 496 | 705 |
| Dichlorodihydrofluorescein Diacetate (DCFH) | 505 | 535 |
| Dihydorhodamine 123 (DHR) | 505 | 535 |
| DsRed | 558 | 583 |
| Europium (III) chloride | 345 | 614 |
| Europium | 345 | 615-620 |
| FL-645 | 615-625 | 665 |
| FITC | 490-494 | 520-525 |
| Fura Red ™ (high pH) | 572 | 657 |
| Genacryl Brilliant Red B | 520 | 590 |
| Genacryl Brilliant Yellow 10GF | 430 | 485 |
| Genacryl Pink 3G | 470 | 583 |
| Genacryl Yellow 5GF | 430 | 475 |
| Green Fluoresencent Protein (GFP) | 498 | 516 |
| LaserPro | 795 | 812 |
| Laurodan | 355 | 460 |
| Leucophor PAF | 370 | 430 |
| Leucophor SF | 380 | 465 |
| Leucophor WS | 395 | 465 |
| Lissamine Rhodamine | 572-577 | 591-592 |
| LOLO-1 | 566 | 580 |
| LO-PRO-1 | 568 | 581 |
| Lucifer Yellow | 425-428 | 528-540 |
| Mag Green | 507 | 531 |
| Maxilon Brilliant Flavin | 450-460 | 495 |
| Mitotracker | 490-578 | 516-599 |
| Nile Red | 515-555 | 559-640 |
| Nuclear Fast Red | 289-530 | 580 |
| Nuclear Yellow | 365 | 495 |
| Oregon Green ™ | 488-514 | 517-526 |
| PE-Cy5 | 488 | 665-670 |
| PE-Cy7 | 488 | 755 |
| | | 767 |
| Phorwite | 360-380 | 430 |
| Phosphine 3R | 465 | 565 |
| PhotoResist | 365 | 610 |
| Phycoerythrin B [PE] | 546-565 | 575 |
| POPO-1 | 433 | 457 |
| PO-PRO-1 | 435 | 455 |
| Procion Yellow | 470 | 600 |
| Rhodamine | 550 | 570 |
| Sevron Brilliant Red | 500-530 | 550-590 |
| Sevron Yellow L | 430 | 490 |
| sgBFP ™ | 387 | 450 |
| Super Glow GFP (sgGFP ™) | 474 | 488 |
| Tetramethylrhodamine (TRITC) | 555 | 576 |
| Texas Red ™ | 595 | 620 |
| Texas Red-X ™ conjugate | 595 | 615 |
| Thiadicarbocyanine (DiSC3) | 651 | 674 |
| | 653 | 675 |
| Thiazine Red R | 596 | 615 |
| Thiazole Orange | 510 | 530 |
| Thioflavin 5 | 430 | 450 |
| Thioflavin S | 430 | 550 |
| Thioflavin TCN | 350 | 460 |
| Thiolyte | 370-385 | 477-488 |
| Thiozole Orange | 453 | 480 |
| TMR | 550 | 573 |
| TO-PRO-1 | 515 | 531 |
| TO-PRO-3 | 644 | 657 |
| TO-PRO-5 | 747 | 770 |
| TOTO-1 | 514 | 531-533 |
| TOTO-3 | 642 | 660 |
| TriColor (PE-Cy5) | 488 | 650, 667 |
| TetramethylRodamineIsoThioCyanate | 550 | 573 |
| True Blue | 365 | 425 |
| TruRed | 490 | 695 |
| Ultralite | 656 | 678 |
| Uranine B | 420 | 520 |
| Uvitex SFC | 365 | 435 |
| X-Rhodamine | 580 | 605 |
| XRITC | 582 | 601 |
| Xylene Orange | 546 | 580 |
| Y66F | 360 | 508 |
| Y66H | 360 | 442 |
| Y66W | 436 | 485 |
| YO-PRO-1 | 491 | 506 |
| YO-PRO-3 | 613 | 629 |
| XL665 | | |
| d2 | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for evaluating the formation or stability of a ternary complex, comprising:
providing a sample that comprises at least three binding members under conditions that allow the formation of the ternary complex to occur;
detecting, quantifying or monitoring a change in the level of the ternary complex using a homogeneous proximity-based detection method,
wherein the formation or stability of the ternary complex is evaluated over a specified time interval, or in the presence of a test agent relative to a reference sample.

2. A homogenous assay for evaluating the formation or stability of a ternary complex, comprising:
providing a sample that comprises at least three binding members under conditions that allow the formation of the ternary complex to occur;
detecting, quantifying or monitoring a change in the level of the ternary complex using a proximity-based detection method,
wherein the formation or stability of the ternary complex is evaluated over a specified time interval, or in the presence of a test agent relative to a reference sample.

3. A method of identifying an agent that modulates the formation or stability of a ternary complex, comprising:
contacting a sample that comprises at least three binding members with a test agent under conditions that allow the formation of the ternary complex to occur;
detecting, quantifying or monitoring the presence of the complex in the sample contacted with the test agent relative to a reference sample using a homogeneous proximity-based detection method,
wherein a change in the level of the complex in the presence of the test agent, relative to the level of the complex in the reference sample, indicates that said test agent affects the formation or stability of said complex.

4. An assay for identifying an agent that modulates the formation or stability of a ternary complex, comprising:
contacting a sample that comprises at least three binding members with a test agent under conditions that allow the formation of the ternary complex to occur;
detecting, quantifying or monitoring the presence of the complex in the sample contacted with the test agent relative to a reference sample using a homogeneous proximity-based detection method,
wherein a change in the level of the complex in the presence of the test agent, relative to the level of the complex in the reference sample, indicates that said test agent affects the formation or stability of said complex.

5. The method of claim 3, wherein the level of the complex in the presence of the test agent decreases relative to the reference sample, said decrease being indicative of a decrease in the formation or stability of the complex.

6. The assay of claim 4, wherein the level of the complex in the presence of the test agent decreases relative to the reference sample, said decrease being indicative of a decrease in the formation or stability of the complex.

7. The method of claim 5, wherein the reference sample is chosen from one or more of a control sample not exposed to the test agent; a control sample exposed to known inhibitor of the complex; or a control sample exposed to an excess amount of an unlabeled binding member of the complex.

8. The assay of claim 6, wherein the reference sample is chosen from one or more of a control sample not exposed to the test agent; a control sample exposed to known inhibitor of the complex; or a control sample exposed to an excess amount of an unlabeled binding member of the complex.

9. The method of either of claims 1 or 3, wherein the at least three binding members comprise a first, second and third binding members, wherein the first binding member is a cytokine, the second binding member is a cytokine receptor and the third binding member is a cytokine co-receptor.

10. The method of claim 9, wherein the cytokine is selected from the group of interleukin 2 (IL-2), interleukin 6 (IL-6), interleukin 4 (IL-4), interleukin 5 (IL-5), interleukin 10 (IL-10), interleukin-13 (IL-13), interleukin 15 (IL-15), interleukin 21 (IL-21) and interleukin 22 (IL-22).

11. The method of claim 9, wherein the cytokine is IL-13, the cytokine receptor is IL-13 receptor α1, and the cytokine co-receptor is IL-4 receptor α.

12. The assay of either of claims 2 or 4, wherein the at least three binding members comprise a first, second and third binding members, wherein the first binding member is a cytokine, the second binding member is a cytokine receptor and the third binding member is a cytokine co-receptor.

13. The assay of claim 12, wherein the cytokine is selected from the group of IL-2, IL-4, IL-5, IL-6, IL-10, IL-13, IL-15, IL-21 and IL-22.

14. The assay of claim 12, wherein the cytokine is IL-13, the cytokine receptor is IL-13 receptor α1, and the cytokine co-receptor is IL-4 receptor α.

15. The method of claim 1, wherein at least one parameter of the assembly, stability, or function of the ternary complex is evaluated, wherein said at least one parameter is selected from the group consisting of kinetics of complex association, kinetics of complex dissociation, binding affinity, and steady-state binding parameters.

16. The method of claim 3, wherein the first binding member is IL-13, the second binding member is IL-13Rα1, and the third binding member is IL-4Rα; and
wherein the test agent interferes with the formation or stability of a binary complex of IL-13 and IL-13Rα1, or interferes with the formation or stability of an interaction between a binary complex of IL-13 and IL-13Rα1, and IL-4Rα.

17. The assay of claim 4, wherein the first binding member is IL-13, the second binding member is IL-13Rα1, and the third binding member is IL-4Rα; and
wherein the test agent interferes with the formation or stability of a binary complex of IL-13 and IL-13Rα1, or interferes with the formation or stability of an interaction between a binary complex of IL-13 and IL-13Rα1 and IL-4Rα.

18. The method of claim 3, further comprising one or more of:
comparing binding of the test agent to the complex to the binding of the known compound to the complex; or
detecting an interaction of the test agent to a complex of two or more of the binding members, relative to the individual members.

19. The assay of claim 4, further comprising one or more of:
comparing binding of the test agent to the complex compared to the binding of the known compound to the complex; or
detecting an interaction of the test agent to a complex of two or more of the binding members, relative to the individual members.

20. The method of claim 10, wherein the formation or stability of the complex is detected by one or more of: a change in the binding or physical formation of the complex itself, a change in signal transduction, or a change in cell function.

21. The assay of claim 13, wherein the formation or stability of the complex is detected by one or more of: a change in the binding or physical formation of the complex itself, a change in signal transduction, or a change in cell function.

22. The method of claim 20, wherein the change in the binding or physical formation of the complex is detected by fluorescence resonance energy transfer (FRET)-based assays or surface plasmon resonance (SPR), wherein the FRET-based assays is chosen from one or more of FRET, Time Resolved FRET assays (TR-FRET), or Bioluminescence Resonance Energy Transfer (BRET).

23. The assay of claim 21, wherein the change in the binding or physical formation of the complex is detected by fluorescence resonance energy transfer (FRET)-based assays or surface plasmon resonance (SPR), wherein the FRET-based assays is chosen from one or more of FRET, Time Resolved FRET assays (TR-FRET), or Bioluminescence Resonance Energy Transfer (BRET).

24. A method for identifying one or more members within a multimeric complex, comprising:
detectably identifying a library of candidate binding members;
detectably identifying at least one known member of the complex;
contacting said identified library with said identified at least one member of the complex, under conditions that allow an interaction to occur,
wherein the interaction of the library member with the at least one member of the complex results in a detectable signal.
